(19) United States
(12) Patent Application Publication
(10) Pub. No.: US 2006/0041947 A1
(43) Pub. Date: Feb. 23, 2006

(54) Nessie nucleic acids, polypeptides and mutations, and methods of use thereof

(76) Inventors: Nancy Ai-Hua Hong, San Diego, CA (US); Aude Fahrer, Canberra (AU); Hua Wu, San Diego, CA (US); Richard J. Glynne, La Jolla, CA (US); Christopher C. Goodnow, Ainslie (AU)

Correspondence Address: FOLEY & LARDNER LLP, P.O. BOX 80278, SAN DIEGO, CA, 92138-0278, US

(21) Appl. No.: 11/171,620

(22) Filed: Jun. 30, 2005

Related U.S. Application Data
(60) Provisional application No. 60/585,163, filed on Jul. 1, 2004.

(51) Int. Cl. A01K 67/027; C07H 21/04; C12P 21/06; C07K 14/47; C07K 16/18; C12N 5/06

(52) U.S. Cl. 800/18; 435/69.1; 435/320.1; 435/325; 530/350; 536/23.5; 435/334; 530/388.1

(57) ABSTRACT

The present invention relates to the discovery, identification, and characterization of novel genes encoding proteins that play a role in immune system development and function, collectively termed Nessie. The invention encompasses the described polynucleotides, the encoded proteins, fusion proteins, polypeptides and peptides, genetically engineered animals that either under- or over-express the disclosed sequences, antibodies to the encoded proteins and peptides, host cell expression systems, antagonists and agonists of the proteins, and other compounds that modulate the expression or activity of the proteins encoded by the disclosed sequences that can be used for diagnosis, drug screening, clinical trial monitoring and the treatment of diseases and disorders.

### CROSS REFERENCE TO RELATED APPLICATIONS

This application claims the benefit of Hong et al. U.S. Provisional Application No. 60/585,163, filed Jul. 1, 2004, which is hereby incorporated herein by reference in its entirety and for all purposes.

### FIELD OF THE INVENTION

The present invention relates to the discovery, identification, and characterization of novel genes and proteins related to immune system function.

### BACKGROUND OF THE INVENTION

The following description of the background of the invention is provided simply as an aid in understanding the invention and is not admitted to describe or constitute prior art to the invention.

T cell development is characterized by stages of proliferation (cell division) and differentiation. The molecules involved in regulation of proliferation during development are often important later in mature T cells for proliferation evoked by antigen-induced activation of the immune response. Furthermore, cells which have undergone abnormal developmental maturation and selection can sometimes exhibit aberrant proliferative qualities as mature T cells. Often these aberrant qualities can lead to immune system disorders such as autoimmunity, cancers, or immunosuppression.

### SUMMARY OF THE INVENTION

The present invention describes the discovery, identification, and characterization of polynucleotides that encode novel proteins, and the corresponding amino acid sequences of these proteins. The novel proteins described for the first time herein have been designated “Nessie.” The analysis of mutations in Nessie proteins described herein confirm that deficits in Nessie function are causative of immune system dysfunction in animals, including mammals.

The understanding of the key role played by Nessie and Nessie-like proteins in normal immune system development indicates that Nessie itself provides a novel diagnostic and therapeutic target. Genetically engineered animals that either under- or over-express the disclosed sequences, antibodies to the encoded proteins and peptides, host cell expression systems, antagonists and agonists of the proteins, and other compounds that modulate the expression or activity of the proteins encoded by the disclosed sequences that can be used for diagnosis, drug screening, clinical trial monitoring and the treatment of diseases and disorders are described herein.

In a first aspect, the present invention relates to nucleic acids encoding wildtype Nessie polypeptides, and Nessie polypeptides. In a related aspect, the present invention relates to nucleic acids encoding mutated Nessie polypeptides, and polypeptides encoded by such nucleic acids. The nucleic acids of the present invention include genomic DNA sequences, RNA sequences, cDNA sequences, introns, exons, etc. In certain preferred embodiments, the novel nucleic acid sequences described herein encode proteins comprising open reading frames (ORFs) of 607 and 605 amino acids in length (see, e.g., SEQ ID NOS: 1 and 2 respectively).

In preferred embodiments, the mutated Nessie nucleic acids encode polypeptides comprising one or more mutations in an amino acid coding sequence that alters the sequence of an expressed protein relative to wildtype. In particularly preferred embodiments, the mutated Nessie nucleic acids produce an altered immune system-related phenotype in an animal expressing the mutated Nessie nucleic acid.

The term “Nessie nucleic acid” as used herein refers to a nucleic acid encoding a Nessie polypeptide, including, but not limited to, a genomic nucleic acid, hnRNA, mature mRNA, cDNA; amplification products thereof; and the Watson-Crick complements thereof. Preferred Nessie nucleic acids are the mouse and human wildtype Nessie nucleic acids having substantial homology to the sequences of SEQ ID NO: 1 and 2. Substantial homology as used herein refers to nucleic acids having at least about 70%, about 75%; more preferably at least about 80%, about 85%; and most preferably about 90%, about 95%, or about 98% identity to an equal length segment of SEQ ID NO: 1 or 2. The term “about” in this context refers to +/−1% of a given measurement. Preferably, Nessie nucleic acids comprise at least 15 consecutive nucleotides, preferably at least 20 consecutive nucleotides, more preferably at least 40 consecutive nucleotides, and most preferably at least 60 consecutive nucleotides of the sequences of SEQ ID NO: 1 and 2. A Nessie nucleic acid, protein, or mutated version thereof, of the present invention may be obtained from numerous species, including humans and non-human animal species such as primate, caprine, bovine, ovine, porcine, and murine species. Most preferably, a Nessie nucleic acid, protein, or mutated version thereof, of the present invention is obtained from a mouse.

The term “altered immune system phenotype” as used herein refers to one or more detectable characteristics of the immune system in an animal that differ from the normal immune system that is typically characteristic in that animal's species.

The terms “expression” and “expressing” as used herein in reference to nucleic acid sequences refers to the translation of a nucleic acid sequence by cellular machinery to provide a polypeptide.

The term “Nessie genomic sequence” as used herein refers to a nucleic acid sequence encoding all or a portion of a Nessie polypeptide in which any intronic and extronic sequences present in the nucleic acid sequence are in the same linear organization as in the Nessie gene from which the Nessie genomic sequence arises. The Nessie gene organization from mouse is described in detail hereinafter.

The term “Nessie polypeptide” as used herein refers to a polypeptide of between about 500 and about 700 amino acids comprising at least one N-terminal cohesin-like domain, bearing similarity to the conserved residues of the Rad21, Rec8 and Scc1 families involved in chromatid cohesion, within a 50-amino acid domain. Preferably, a Nessie polypeptide is at least about 80% or about 90%, preferably at least about 95%, and most preferably at least about 98% identical to an equal length segment of a wildtype Nessie polypeptide. The term “about” in this context refers to +/−1% of a given measurement. The terms “wildtype mouse Nessie polypeptide” and “wildtype human Nessie polypeptide” as used herein refers to the mouse and human wildtype Nessie polypeptides having the sequences of SEQ ID NOs: 5 and 6.

The present invention also relates to fragments of such Nessie nucleic acids and polypeptides. Preferably, a Nessie nucleic acid fragment comprises at least 15 consecutive nucleotides, preferably at least 20 consecutive nucleotides, more preferably at least 40 consecutive nucleotides, and most preferably at least 60 consecutive nucleotides of a Nessie nucleic acid; and a Nessie polypeptide fragment comprises at least 5 consecutive amino acid residues, preferably at least 7 consecutive amino acid residues, more preferably at least 10 consecutive amino acid residues, even more preferably at least 15 consecutive amino acid residues, and most preferably at least 20 consecutive amino acid residues of a Nessie polypeptide.

The term “mutated Nessie nucleic acid” as used herein refers to a nucleic acid encoding a mutated Nessie polypeptide, including, but not limited to, a mutated Nessie genomic sequence, a mutated hnRNA, a mutated mature mRNA, a mutated cDNA; amplification products thereof; and the Watson-Crick complements thereof.

The term “mutated Nessie polypeptide” refers to a polypeptide that is at least about 90%, preferably at least about 95%, and most preferably at least about 98% identical to an equal length segment of a wildtype Nessie polypeptide obtained from the same species. The term “about” in this context refers to +/−1% of a given measurement. Protein identity is determined by aligning two sequences using BLAST (Altschul, et al., “Gapped BLAST and PSI-BLAST: a new generation of protein database search programs.” Nucleic Acids Res. 25:3389-3402(1997)) with default parameters, and determining the number of identical amino acids relative to the total length of the mutated Nessie polypeptide. Preferably, a mutated Nessie polypeptide produces an altered immune system-related phenotype when expressed in an animal.

The present invention also relates to fragments of such mutated Nessie nucleic acids and polypeptides. Preferably, a mutated Nessie nucleic acid fragment comprises at least 15 consecutive nucleotides, preferably at least 20 consecutive nucleotides, more preferably at least 40 consecutive nucleotides, and most preferably at least 60 consecutive nucleotides of a mutated Nessie genomic sequence, hnRNA, mature mRNA, cDNA, or the complement thereof, which includes the site of the mutation; and a mutated Nessie polypeptide fragment comprises at least 5 consecutive amino acid residues, preferably at least 7 consecutive amino acid residues, more preferably at least 10 consecutive amino acid residues, even more preferably at least 15 consecutive amino acid residues, and most preferably at least 20 consecutive amino acid residues of a mutated Nessie polypeptide, which includes the site of the mutation.

While mutations in the mutated Nessie nucleic acids and polypeptides of the present invention may include deletions, substitutions, inversions, insertions, etc., it is preferred that the nucleic acids of the present invention encode polypeptides that are reduced in length to their corresponding wildtype counterparts; most preferably, the mutation is caused by an alteration in an intronic sequence, resulting in altered RNA size (e.g., a mis-splicing event) relative to a wildtype RNA.

In another aspect, the present invention relates to one or more transgenic animals that comprise one or more non-wildtype Nessie genes. The skilled artisan will understand that a gene is present in a diploid genome as two alleles. In various embodiments, such transgenic animals may comprise a mutated Nessie transgene in addition to or in place of one or both wildtype alleles; or may be “knockout” animals (which can be conditional) that do not express a functional Nessie or have reduced levels of Nessie expression. Animals may also comprise endogenous Nessie nucleic acids and/or mutated Nessie nucleic acids. The term “endogenous” as used herein in reference to cells refers to a nucleic acid sequence that is present in cells without the use of recombinant DNA techniques. Various animal cells will naturally contain Nessie nucleic acid sequences and/or mutated Nessie nucleic acid sequences.

The invention provides transgenic non-human animals comprising a nucleic acid, a polypeptide, an expression cassette or vector or a transfected or transformed cell of the invention. The transgenic non-human animals can be, e.g., goats, rabbits, sheep, pigs, cows, rats and mice, comprising the nucleic acids of the invention. A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats.

The invention provides transgenic non-human animals that do not express their endogenous Nessie polypeptides, or, express their endogenous Nessie polypeptide at lower than wild type levels (thus, while not completely “knocked out” their Nessie activity is functionally “knocked out”). The invention also provides “knockout animals” and methods for making and using them. For example, in one aspect, the transgenic or modified animals of the invention comprise a “knockout animal,” e.g., a “knockout mouse,” engineered not to express an endogenous gene, e.g., an endogenous Nessie gene, which is replaced with a gene expressing a polypeptide of the invention, or, a fusion protein comprising a polypeptide of the invention. Thus, in one aspect, the inserted transgenic sequence is a sequence of the invention designed such that it does not express a functional Nessie polypeptide. The defect can be designed to be on the transcriptional, translational and/or the protein level. Because the endogenous Nessie gene has been “knocked out,” only the inserted polypeptide of the invention is expressed.

A “knock-out animal” is a specific type of transgenic animal having cells that contain DNA containing an alteration in the nucleic acid sequence that reduces the biological activity of the polypeptide normally encoded therefrom by at least 80% compared to the unaltered gene. The alteration may be an insertion, deletion, frameshift mutation, missense mutation, introduction of stop codons, mutation of critical amino acid residue, removal of an intron junction, and the like. Preferably, the alteration is an insertion or deletion, or is a frameshift mutation that creates a stop codon. Typically, the disruption of specific endogenous genes can be accomplished by deleting some portion of the gene or replacing it with other sequences to generate a null allele. Cross-breeding mammals having the null allele generates a homozygous mammals lacking an active copy of the gene.

A number of such mammals have been developed, and are extremely helpful in medical development. For example, U.S. Pat. No. 5,616,491 describes knock-out mice having suppression of CD28 and CD45. Procedures for preparation and manipulation of cells and embryos are similar to those described above with respect to transgenic animals, and are well known to those of ordinary skill in the art.

Endogenous Nessie nucleic acid sequences may be mutated, e.g., using mutagens such as: radiation (gamma, beta, alpha, UV, etc.); base analogues such as bromouracil and aminopurine; chemicals such as nitrous acid, nitrosoguanidine, ethylnitrosourea, and ethylmethanesulfonate; intercalating agents such as acridine orange and ethidium bromide, to provide endogenous mutated Nessie family nucleic acid sequences. In exemplary embodiments, the present invention describes a mouse line that carries a mutated version of the endogenous Nessie gene resulting in an altered immune system-related phenotype. Exemplary methods for providing endogenous mutated Nessie nucleic acid sequences are described hereinafter.

The animals of the present invention may be used for a variety of purposes, including the production of proteins encoded by expression constructs, screening methods to identify modulators of Nessie polypeptides, testing the effects of such modulators on altered immune system-related phenotypes, identifying genetic modulators of altered immune-related phenotypes, sensitized screening methods, etc.

In another aspect, the present invention relates to agonists and antagonists of Nessie protein, including small molecules (i.e., molecules, particularly organic molecules, having a molecular weight less than about 2000, 1500, 1000, 750, or even 500 Dalton), large molecules (i.e., molecules of molecular weight greater than about 2000 Dalton), mutant Nessie proteins, or portions thereof that compete with native proteins, peptides, and antibodies, as well as nucleotide sequences that can be used to inhibit the expression of Nessie proteins (e.g., antisense, ribozyme, decoy oligos, and RNAi molecules, and gene or regulatory sequence replacement constructs) or to enhance the expression of Nessie proteins (e.g., expression constructs that place the described sequences under the control of a strong promoter system).

Further, the present invention also relates to processes for identifying compounds that modulate, i.e., act as agonists or antagonists, of Nessie expression and/or Nessie activity that utilize purified preparations of the Nessie product, or cells expressing the same. Such compounds can be used as therapeutic agents for the treatment of any of a wide variety of symptoms associated with biological disorders or imbalances.

In another aspect, the present invention relates to proteins that interact with the Nessie protein and modulate or are modulated by Nessie protein activity. Such proteins can be identified by methods which may include available in vivo or in vitro based screening systems to detect the interactions between two proteins. For instance, commonly available genetic systems are capable of rapidly detecting which proteins interact with a known protein, determining which domains of the proteins interact and identifying agents which modulate the interaction between two proteins. One such system is the yeast two-hybrid system wherein two proteins are expressed in yeast: one protein of interest fused to a DNA-binding domain and the other protein of interest fused to a transcriptional activation domain (Fields (1989) Nature 340(6230):245-246).

In another aspect, the present invention relates to recombinant DNA vectors comprising a Nessie nucleic acid, and/or a mutated Nessie nucleic acid. Preferably, such a vector is an expression construct in which a Nessie nucleic acid, and/or a mutated Nessie nucleic acid, is operably inserted downstream in the direction of transcription of a transcriptional regulatory region functional in a cellular expression host. Vectors, plasmids or viruses, may be used to prepare such recombinant DNA vectors. In preferred embodiments, recombinant DNA vectors of the present invention will usually have a gene for positive selection, e.g. antibiotic, enzyme that produces a detectable substrate, etc., and may have other features known to those of skill in the art for integration or permanent maintenance, i.e. replication.

The term “recombinant DNA vector” as used herein refers to a circular or linear DNA molecule that contains an inserted piece of DNA, and that is capable of replication in certain host cells. A recombinant DNA vector may be replicated without integration into the host cell genome, or may be integrated into the genome. Such vectors may be capable of replicating in prokaryotic cells, in eukaryotic cells, or both. Reco promoter which is capable of initiating the 5′ synthesis of RNA from cDNA is selected from the group consisting of the MoMLV promoter, metallothionein promoter, glucocorticoid promoter, SV 40 promoter, and the CMV promoter recombinant DNA vectors are well known to those of skill in the art. See, e.g., U.S. Pat. Nos. 6,391,585; 6,342,372; 6,326,195; 6,210,939; 6,057,152; 5,665,578; 5,646,009; and 5,604,118.

Preferred recombinant DNA vectors of the present invention comprise a nucleic acid sequence encoding a mouse wildtype Nessie polypeptide having the sequence of SEQ ID NO: 5, most preferably encoded by the nucleic acid of SEQ ID NO: 1. Other preferred recombinant DNA vectors of the present invention comprise a nucleic acid sequence encoding a mutated Nessie polypeptide having the sequence of SEQ ID NO: 3.

The term “expression construct” as used herein refers to a recombinant DNA vector comprising regulatory regions providing for transcription and translation of an inserted piece of DNA when propagated in suitable host cells. In addition to an origin of replication and a selection gene, an expression construct typically contains a promoter, a ribosome binding site, and a transcription terminator. Suitable promoters include both eukaryotic and prokaryotic promoters such as, for example, P-galactosidase promoter, trpE promoter, lacZ promoter, T7 promoter, T3 promoter, SP6 promoter, the MoMLV promoter, metallothionein promoter, glucocorticoid promoter, SV 40 promoter, and the CMV promoter. Additional optional elements, such as enhancers, multiple cloning sites, etc., may also be present.

In another aspect, the recombinant DNA vectors of the present invention may be inserted into suitable eukaryotic or prokaryotic cells to provide “host cells” that comprise a Nessie nucleic acid or mutated Nessie nucleic acid, e.g., using transfection or transformation techniques well known to those of skill in the art. Eukaryotic host cells may be obtained or derived from numerous animal species, including humans and non-human animal species such as primate, caprine, bovine, ovine, porcine, and murine species.

Preferably, such host cells are cells, other than an intact animal, comprising one or more introduced Nessie nucleic acids; and cells, other than an intact animal, comprising one or more introduced mutated Nessie nucleic acids. In particularly preferred embodiments, such cells express the introduced nucleic acid.

The term “introduced” as used herein in reference to cells refers to a nucleic acid sequence inserted into one or more cells by recombinant DNA techniques. An introduced Nessie nucleic acid may be, for example, a Nessie nucleic acid obtained from one species and introduced into a cell of a different species, such as a nucleic acid encoding a human Nessie protein inserted into a mouse cell or vice versa. Alternatively, an introduced Nessie nucleic acid encoding a human Nessie protein may be inserted into a human cell, or a nucleic acid encoding a mouse Nessie protein may be inserted into a mouse cell, using recombinant DNA techniques. Preferably, an introduced Nessie nucleic acid is in a recombinant DNA vector, and the vector is inserted into a cell.

Host cells prepared according to the present invention may be used for a variety of purposes, including ex vivo gene transfer methods, the production of proteins encoded by expression constructs, screening methods to identify modulators of Nessie polypeptides, etc.

Suitable cells for use in such methods include host cells comprising one or more introduced wildtype or mutated Nessie nucleic acids, as described herein, and/or cells comprising one or more endogenous wildtype or mutated Nessie nucleic acids. Cells may be contacted in an in vitro environment (e.g., in a cell culture), or in an in vivo environment (e.g., cells in a live fetus or animal). Typically, the presence, amount, or function of endogenous wildtype or mutated Nessie nucleic acids in or from cells treated with the composition(s) will be compared to one or more control cells not so treated. Preferably, both cells to be tested and control cells express the wildtype or mutated Nessie nucleic acid(s) constitutively.

In these cell-based screening methods, the skilled artisan will understand that a test composition may affect the characteristics of the polypeptide at a number of levels, including transcription of the Nessie nucleic acid(s), translation of Nessie nucleic acid(s), or the activity of Nessie polypeptide produced. Additionally, such screening methods may rely on directly measuring the presence, amount, or activity of the wildtype or mutated Nessie DNA, RNA, or protein; or may rely on indirect measurements.

In particularly preferred embodiments, suitable test compositions include those having a direct effect on the properties of a mutated Nessie polypeptide, such as double-stranded RNA designed to provide gene silencing of the mutated Nessie nucleic acid(s) by RNA interference (“RNAi”) (see, e.g., Paddison et al., Proc. Nat'l Acad. Sci. USA 99: 1443-8 (2002); and Hutvagner and Zamore, Curr. Opin. Genet. Dev. 12: 225-32 (2002)); antisense nucleic acids designed to inhibit expression of the mutated Nessie nucleic acid(s) (see, Bavisotto, J. Exp. Med. 174: 1097-1101 (1991)); gene therapy constructs designed to disrupt a Nessie gene (“knockout” constructs); gene therapy constructs designed to overexpress Nessie nucleic acid(s), thereby compensating for the presence of mutated Nessie polypeptides; gene therapy constructs designed to overexpress Nessie nucleic acid(s), thereby compensating for phenotypes related to Nessie polypeptide function; decoy oligonucleotides designed to bind to a mutated Nessie polypeptide but not to a wildtype Nessie polypeptide (see, Mann and Dzau, J. Clin. Invest. 106: 1071-75 (2000)); or a combination of any of these compositions. This list is not mean to be limiting, and other appropriate test compositions will be apparent to those of skill in the art.

Numerous species, including a humans and non-human animal species such as primate, caprine, bovine, ovine, porcine, and murine species, may be used in such screening methods. In preferred embodiments where screening is performed in vivo, the animals used are mice. In yet other preferred embodiments, the effect of such compositions on the immune system is determined.

Preferably, the cells and/or animals used in the screening methods of the present invention comprise a nucleic acid sequence encoding a Nessie polypeptide, most preferably having the sequence of SEQ ID NOs: 5 or 6. Preferred nucleic acids encoding such polypeptides are those having the sequences of SEQ ID NOs: 1 or 2.

In another aspect, the present invention relates to methods of identifying mutations in a Nessie gene. Such mutations may produce a dominant or recessive immune system-related phenotype in an animal. These methods comprise identifying one or more animals exhibiting such a dominant or recessive altered phenotype, and determining whether a mutated Nessie gene is present in said animal.

A phenotype is said to be “recessive” if the relevant characteristics detectable in a homozygotic animal are not apparent in an animal that is heterozygotic for the mutation of interest. A phenotype is said to be “dominant” if the relevant characteristics are detectable in a heterozygotic animal.

Numerous animal species, including humans and non-human animal species such as primate, caprine, bovine, ovine, porcine, and murine species, may be used for identifying mutations in a Nessie gene that produce a recessive altered immune system-related phenotype according to the present invention. In preferred embodiments, the animals used are mice.

Once one or more animals exhibiting a recessive altered immune system-related phenotype are identified, Nessie gene sequences can be determined using sequencing methods that are well known to those of skill in the art. In preferred embodiments, mutations are identified by comparison to the wildtype Nessie nucleic acid having the sequence of SEQ ID NOs: 1 or 2. Alternatively, Nessie mRNA, cDNA, and/or polypeptides may be sequenced. In preferred embodiments, Nessie mutations can be identified by comparison to proteins having the sequence of SEQ ID NOs: 5 or 6.

In another aspect, the present invention relates to methods of identifying subjects in need of compositions modulating one or more characteristics of mutated Nessie polypeptides, and methods for administering such compositions to a subject. The methods comprise contacting one or more cells of the subject expressing mutated Nessie polypeptides with one or more compositions that affect one or more characteristics of the mutated polypeptide.

The term “subject” as used herein refers to a human or a non-human animal. Thus, the methods and compositions described herein can be used for both medical and veterinary purposes.

A subject may be identified as being in need of administration of one or more compositions that affect one or more characteristics of the mutated Nessie polypeptides by numerous methods well known to those of skill in the art. For example, the presence, amount, or activity of the mutated Nessie genomic DNA, immature or mature messenger RNA, or expressed protein may be determined.

Alternatively, or together with determining mutated Nessie DNA, RNA, or protein, indirect measurements, such as identification of an altered immune system-related phenotype in a subject may also be used to identify suitable subjects. Preferably, administration of such composition(s) ameliorates one or more of these phenotypes, i.e., returns an aberrant phenotype to a normal or increasingly normal phenotype.

Such methods may advantageously be used to diagnose patients suffering from or at risk for immune system-related diseases, or to identify homozygous carrier states, and for treatment of patients with compounds that have an effect on immune system function. The skilled artisan will further understand that such methods may also be used to monitor the course of treatment of a subject with compositions that affect one or more characteristics of the mutated Nessie polypeptides. For example, T cell numbers in such a subject may be monitored for possible improvement by such treatment.

In another aspect, the present invention relates to kits for determining the presence or amount of a mutated Nessie DNA, RNA, or protein in a sample. Such kits preferably comprise one or more assay components for detecting the mutated Nessie DNA, RNA, or protein, and may optionally include one or more of: instructions for performing the detection; reagents, such as buffers, for use in performing the detection; pipettes for liquid transfers; etc.

A suitable assay component may comprise an antibody, or a fragment or variant thereof, may be provided that is capable of specifically binding mutated Nessie. A method of raising the antibody preferably comprises isolating the antibody from an animal or isolating an antibody-producing cell from an animal, following administration of mutated Nessie protein, or an antigenic fragment thereof, to the animal. Additional methods for obtaining antibody-like molecules, such as single chain variable region fragments obtained from phage display, are also well known to those of skill in the art. An antibody of the invention may be useful in detecting or measuring the presence of mutated Nessie protein in an individual, by contacting the antibody with a biological sample from a subject.

The term “specifically binds” as used herein with regard to antibodies does not indicate that there is no binding of the antibody to non-target protein(s). Rather, an antibody is defined as being “specific for”, as “specifically reacting with”, or as “specifically binding to”, target protein(s) if the antibody exhibits a binding affinity for target protein that is at least about twice the affinity exhibited for a non-target protein. Certain preferred antibodies exhibit a binding affinity for the mutated Nessie protein of interest that is at least about twice the affinity exhibited for binding to non-Nessie proteins. Other preferred antibodies exhibit a binding affinity for the mutated Nessie protein of interest that is at least about twice the affinity exhibited for a Nessie protein lacking the mutation.

A suitable assay component may also comprise a nucleic acid that hybridizes under stringent conditions to a mutated Nessie nucleic acid of interest, but that does not hybridize under such conditions to a Nessie nucleic acid lacking the mutation. Such “probe” nucleic acids are at least 15 nucleotides in length, more preferably at least 20 nucleotides in length, and even more preferably 30 nucleotides in length or more, and contain nucleotide(s) corresponding to the mutation of interest.

Such “probe” nucleic acids may be substantially complementary to a mutated Nessie nucleic acid of interest. By “substantially complementary” is meant that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target sequence, positioned 3′ or 5′ to a contiguous sequence of bases that hybridize under stringent hybridization conditions to a target sequence.

The term “stringent hybridization conditions” refers to 42° C. in 50% formamide, 5×SSPE, 0.3% SDS, and a repetitive sequence blocking nucleic acid, such as cot-1 or salmon sperm DNA (e.g., 200 ng/ml sheared and denatured salmon sperm DNA).

A suitable assay component may also comprise a nucleic acid suitable for use in a primer extension assay for detection of a mutated Nessie nucleic acid of interest. As used herein, “primer extension” refers to the enzymatic extension of the three-prime (3′) hydroxy group of an extension primer, which is an oligonucleotide that is paired in a duplex to a template nucleic acid. For an example of primer extension as applied to the detection of polymorphisms, see Fahy et al., Multiplex fluorescence-based primer extension method for quantities mutation analysis of mitochondrial DNA and its diagnostic application for Alzheimer's disease, Nucleic Acid Research 25:3102-3109, 1997. The extension reaction is catalyzed by a DNA polymerase. Extension of the 3′ end of the oligonucleotide generates an oligonucleotide having a length greater than the extension primer and having a sequence that is the reverse complement of the template nucleic acid. If one of the nucleotides in the added sequence is labeled, then the extended oligonucleotide becomes labeled. Extension primers must be of a length sufficient to provide specific binding to the target sequence of interest. The extension primer sequence has a 3′ terminus that pairs with a nucleotide base that is, in the sample nucleic acid to which the primer is hybridized, 5′ from the site of one or more bases in the sequence of interest that represent a mutation of interest.

By “DNA Polymerase” it is meant a DNA polymerase, or a fragment thereof, that is capable of catalyzing the addition of bases to a primer sequence in a sequence-specific fashion. A DNA polymerase can be an intact DNA polymerase, a mutant DNA polymerase, an active fragment from a DNA polymerase, such as the Klenow fragment of E. coli DNA polymerase, and a DNA polymerase from any species, including but not limited to thermophilic organisms.

The term “biological sample” as used herein refers to a sample obtained from a cell, tissue, or organism. Examples of biological samples include proteins and/or nucleic acids obtained from cells (e.g., mammalian cells, bacterial cells, cultured cells), particularly as a lysate, a biological fluid (such as blood, plasma, serum, urine, bile, saliva, tears, cerebrospinal fluid, aqueous or vitreous humor, or any bodily secretion), a transudate or exudate (e.g., fluid obtained from an abscess or other site of infection or inflammation), a fluid obtained from a joint (e.g., a normal joint or a joint affected by disease such as rheumatoid arthritis, osteoarthritis, gout or septic arthritis), or the like.

The summary of the invention described above is not limiting and other features and advantages of the invention will be apparent from the following detailed description of the preferred embodiments, as well as from the claims.

### BRIEF DESCRIPTION OF FIGURES

FIG. 1 is a schematic of the genomic locus of mouse Nessie gene with coordinates based on the public Mouse Genome Sequencing Consortium NCBI_M30 (February, 2003) genomic assembly.

FIG. 2 shows the results of RT-PCR analysis of wildtype and mutant Nessie transcript in mouse whole thymus RNA. M: marker, 1-5: fragments spanning the Nessie transcript, G: GAPDH control, −: Nessie mutant, +: wildtype. No significant difference in amount of transcript amplification is noted, but a slightly decreased size shift is apparent for fragments 3 and 4 of the mutant Nessie transcript. Sequence analysis of these fragments suggested amplification of non-Nessie sequence, possibly due to lack or reduced levels of wildtype Nessie transcript. Fragment 5 shows multiple bands that could reflect alternative splicing of the gene or possibly cross-priming with other closely related transcripts.

FIG. 3 contains sequencing data of the wildtype and mutant mouse Nessie genes.

FIG. 4 collectively shows the abnormal T cell profile of mice homozygous for the mutant Nessie gene. FIG. 4A shows the results of FACS analysis of bone marrow cells that were collected from wildtype or Nessie mice and used to reconstitute an irradiated wildtype host, thereby testing the intrinsic developmental properties of Nessie thymocytes. A decreased proportion of CD4⁺ CD8⁺ double-positive thymocytes and CD4⁺ and CD8⁺ single-positive thymocytes indicates a partial block in T cell development. FIG. 4B shows the results of FACS analysis of peripheral blood lymphocyte, which shows a clear deficit in mature CD8⁺ T cells in Nessie mice. Remaining CD8⁺ cells are CD44^{high}.

FIG. 5A presents cell number (+SEM) in thymocyte subsets, proceeding from the least mature progenitors on the left to the most mature subsets on the right. The symbol “**” indicates that p<0.01 by two-tailed Student's t-test. Cell subset distribution was determined by flow cytometry with αCD4-FITC, αCD44-TC, αCD25-PE and αCD8-FITC (for DN subpopulations) or αCD8-PE (for other populations). FIG. 5B presents representative histograms showing binding of Annexin V-FITC or anti-CD5-FITC on CD4⁺ CD8⁺ DP thymocytes. Single cell suspensions were stained with fluorochrome-conjugated antibodies (obtained from BD PharMingen). Samples were collected on FACSort or LSRII flow cytometers (Becton Dickinson). Data were analysed using FloJo software (Treestar).

FIG. 6A presents the hemopoietic systems of lethally irradiated B6.5JL.Ly5^{a} mice which were reconstituted with bone marrow cells from a wild-type or nes/nes C57BL6 (Ly5^{b}) mouse, or a 50/50 mixture of bone marrow from nes/nes C57BL6 (Ly5^{b}) and wild type B6.SJL.Ly5^{a} mice. The left three panels are gated on Ly5^{b} spleen cells, showing the subsets of CD44^{hi} and CD44^{low} CD8 cells. The right panel is gated on Ly5^{a} spleen cells in the 50/50 chimeras, showing normal CD8 subsets derived from wt precursors which contrast with the absence of CD44low CD8 cells from nes/nes precursors. Bone marrow chimaeras were performed as previously described ²².(Miosge, L. A., et al., 2002, J Exp Med 196:1113-9). Mice were injected with 1.5-2×10⁶ bone marrow cells i.v., and analysed 3-5 months after reconstitution. FIG. 6B presents hemopoietic chimeras reconstituted with B6 nes/nes marrow as in (FIG. 6A), except that the hemopoietic stem cells were first transduced in vitro with a bicistronic retrovirus expressing the wild type long cDNA from ENSMUSG00000008690 and green fluorescent protein (GFP). Profiles are gated on Ly5b⁺ CD8⁺ nes/nes splenic lymphocytes, and display CD44^{low} and CD44^{hi} subsets of CD8 cells differentiated from transduced (GFP⁺) and non-transduced (GFP⁻) nes/nes stem cells. Panels show data from three independently reconstituted mice in two separate transduction experiments. The MSCV-LTR transfection method has previously been described (Vinuesa, C. G. et al. 2005, Nature 435:452-8). Mice were analysed six weeks after reconstitution. FIG. 6C presents a clustal W alignment of kleisin β sequences from different organisms, showing sequence conservation of the Ile residue mutated in nessie. Numbers in brackets refer to NCBI gi accession numbers. Star, colon and full stop indicate identity, conservation or semi-conservation of amino acids at each position, respectively.

### DETAILED DESCRIPTION OF THE INVENTION

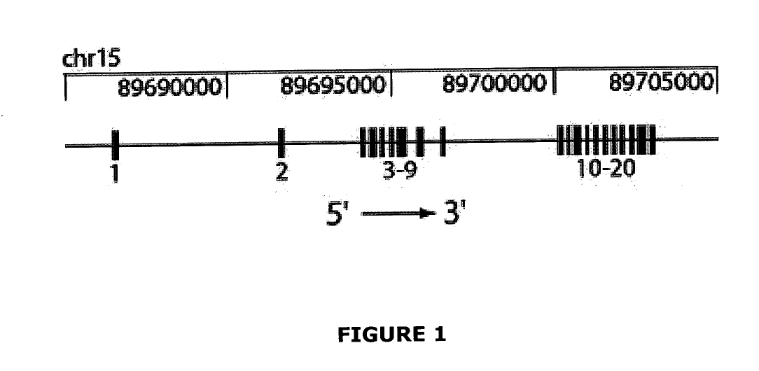

In the present invention, the Nessie gene has been identified in the genomes of many organisms, all of which encode Nessie proteins of between about 500 and about 700 amino acids that contain a N-terminal cohesin-like domain. The mouse Nessie gene locus is located on chromosome 15 and contains at least 20 exons (see FIG. 1).

In addition, a mouse strain exhibiting immune system dysfunction was identified from a library of ethylnitrosourea (ENU) mutagenized mice. This strain carries a point mutation in the Nessie gene. The mutation occurs in a sequence that encodes a well-conserved coding nucleotide in exon 1 of the Nessie gene, leading to an amino acid change in the Nessie protein.

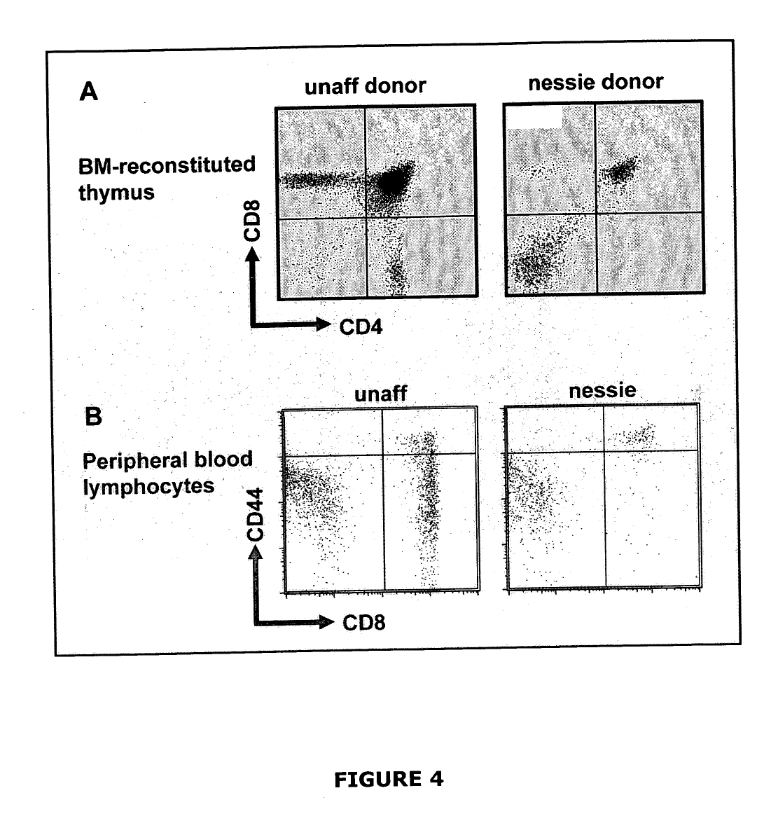

Mice with two copies of a chemically-induced mutation in the Nessie gene are born at an expected rate and exhibit normal growth and behavior. Peripheral blood lymphocytes from homozygous Nessie mice show a clear reduction in frequency of T cells, and characterization of the thymus indicates a partial block in T cell development during the CD4⁻CD8⁻ double negative stage leading to fewer CD4⁺ and CD8⁺ single positive cells (see, e.g., FIG. 4). Mice heterozygous for this mutant allele appear healthy and normal.

Nessie Sequences

Wild type mouse Nessie nucleic acid has the following sequence (SEQ ID NO: 1) (start and stop codons are underlined):

GCCAGAACCAGTAGCGGGTTGAGCCATGGTTCCGGGCTTCTAGTCCCGTT
CTAGACATGGAGGATGTGGAGGTGCGCTTTGCTCACCTCTTGCAGCCCAT
CCGGGATCTCACTAAGAACTGGGAGGTGGACGTGGCGGCACAGCTGGGCG
AGTATCTGGAGGAGCTGGACCAGATCTGCATTTCTTTTGATGAAGGCAAA
ACCACAATGAACTTCATTGAGGCAGCACTGTTGATCCAGGGCTCAGCCTG
TGTCTACAGTAAGAAGGTGGAGTACCTCTACTCGCTGGTCTACCAGGCTC
TCGATTTTATTTCTGGCAAGAGGCGGGCCAAACAGCTCTCCTTAGTTCAG
GAAGATGGGAGCAAGAAGACTGTCAACTCAGAGACTCCCTGTGAAACAGA
GAATGAGTTCCTGTCACTGGACGACTTCCCTGACTCCCGGGCTAATGTGG
ATCTGAAAAATGATCAGGCATCCAGTGAGCTGCTTATCATACCCCTACTG
CCCATGGCCCTGGTGGCCCCTGATGAAGTGGAAAAGAACAGCAGCCCCTT
GTATAGCTGTCAGGGTGACATCTTGGCCAGCCGGAAGGATTTCAGGATGA
ACACGTGTATGCCTAACCCCAGAGGCTGCTTTATGTTAGATCCAGTGGGA
ATGTGTCCTGTGGAGCCTGTGGTGCCCGTGGAGCCATACCCCATGTCAAG
GAGCCAGAAAGATCCTGAGGACGCTGAGGAGCAGCCCATGGAAGTGTCTA
GGAACGGGAGTCCTGTTCCTGTACCCGACATCTCCCAAGAGCCAGATGGT
CCAGCGCTCAGCGGTGGAGAGGAGGATGCAGAGGATGGAGCAGAGCCCCT
GGAGGTTGCTCTAGAGCCTGCAGAGCCAAGGACCTCACAGCAGAGTGCCA
TCTTGCCAAGGAGATACATGCTGCGGGAACGACAAGGGGCACCGGAGCCT
GCCTCCCGGCTACAGGAGACCCCAGACCCCTGGCAGAGCCTGGACCCTTT
TGACTCCTTGGAATCTAAGGTCTTCCAGAAAGGGAAACCCTATTCTGTGC
CACCCGGTGTGGAGGAGGCTCCAGGACAGAAGCGCAAGAGGAAGGGTGCC
ACCAAGTTGCAGGACTTCCACAAGTGGTACCTGGATGCCTATGCTGAACA
CCCTGACGGCAGGAGGGCTCGGCGGAAGGGCCCAACCTTTGCAGACATGG
AAGTCCTGTACTGGAAACATGTGAAAGAACAGCTCGAGACCCTTCAGAAG
CTGCGGAGACGTAAGATCAACGAGAGATGGCTACCTGGGGCCAAGCAGGA
TCTGTGGCCTACAGAGGAGGATCGCTTGGAAGAGTCCCTCGAGGACCTCG
GGGTAGCAGATGACTTTCTAGAGCCCGAGGAGTACGTGGAGGAGCCTGCG
GGGGTGATGCCCGAGGAAGCTGCTGACCTCGATGCAGAGGCCATGCCAGA
GTCCCTGAGATACGAGGAGCTGGTCCGGAGAAATGTGGAACTCTTCATTG
CCACCTCCCAGAAGTTTATCCAGGAGACAGAGCTGAGCCAACGCATCAGG
GACTGGGAAGATACCATCCAGCCCCTGCTCCAGGAGCAGGAGCAGCATGT
GCCCTTTGATATCCATATCTACGGGGACCAGTTGGCTTCACGGTTCCCCC
AGCTCAATGAATGGTGTCCCTTTTCAGAGCTTGTAGCAGGGCAGCCTGCT
TTTGAGGTGTGCCGCTCCATGCTGGCCTCCTTGCAACTGGCTAATGACTA
CACAGTGGAGATCACTCAGCAGCCAGGACTGGAGGCAGCTGTGGACACAA
TGTCTCTGAGACTGCTCACACACCAGCGAGCCCACACCCGCTTCCAGACC
TATGCTGCACCATCCATGGCCCAGCCTTGAGTGGACAGCACTGAGGCAGG
GGTGGAAAGTAGTATATACCTGGAGGTCTTTGCCCCTAATGTGCTATGGG
GCCATTCACTCCAGTGCTGCCTCCTGGCTGGCCTAGCCTAATA

Wild type human Nessie nucleic acid has the following sequence (SEQ ID NO: 2) (start and stop codons are underlined):

GAACTAGTGGCGGGCTGAGGACGCCGTACCCCTCGGAAGGCAGCCCTGCG
GTCCCTTTGCCGCCCGTTCCCTCCCGGACATGGAGGACGTGGAGGCGCGC
TTCGCCCACCTCTTGCAGCCCATCCGCGACCTCACCAAGAACTGGGAGGT
GGACGTGGCGGCCCAGCTGGGCGAGTATCTGGAGGAGCTGGATCAGATCT
GCATTTCTTTTGACGAAGGCAAGACCACAATGAACTTCATTGAGGCAGCG
TTGTTGATCCAGGGCTCTGCCTGCGTCTACAGTAAGAAGGTGGAATACCT
CTACTCACTCGTCTACCAGGCCCTTGATTTCATCTCTGGAAAGAGGCGGG
CCAAGCAGCTCTCTTCGGTGCAGGAGGACAGGGCCAATGGGGTTGCCAGC
TCCGGGGTCCCCCAGGAGGCAGAGAATGAGTTCCTGTCGCTGGATGACTT
CCCTGACTCCCGGACTAACGTGGATCTCAAGAATGATCAGACGCCCAGTG
AGGTCCTCATCATCCCCCTCCTGCCCATGGCCCTGGTGGCCCCTGATGAA
ATGGAGAAGAACAACAATCCCCTGTACAGCCGTCAGGGTGAGGTCCTGGC
CAGCCGGAAGGATTTCAGGATGAACACGTGCGTTCCCCACCCCAGAGGGG
CCTTCATGTTGGAGCCAGAGGGCATGTCCCCCATGGAACCAGCGGGCGTT
TCCCCCATGCCAGGGACCCAGAAGGACACCGGGAGGACTGAGGAGCAGCC
AATGGAAGTTTCCGTGTGCAGGAGCCCTGTCCCAGCACTCGGCTTCTCCC
AGGAGCCAGGCCCCTCTCCAGAAGGCCCGATGCCCCTGGGTGGGGGCGAG
GACGAGGATGCAGAGGAGGCAGTAGAGCTTCCTGAGGCCTCGGCCCCCAA
GGCCGCTCTGGAGCCCAAGGAGTCCAGGAGCCCGCAGCAGAGTGCTGCCC
TGCCCAGGAGGTACATGCTGCGGGAGCGGGAGGGGGCCCCAGAGCCTGCA
TCCTGCGTGAAGGAGACTCCAGACCCCTGGCAGAGCCTGGACCCCTTTGA
CTCCTTGGAGTCTAAGCCCTTCAAGAAAGGTAGGCCTTACTCTGTGCCCC
CCTGTGTGGAGGAGGCTCTGGGACAGAAGCGCAAGAGGAAGGGCGCTGCC
AAGCTGCAGGACTTCCACCAGTGGTACCTGGCTGCCTATGCAGACCATGC
CGACAGCAGGCGGCTTCGGCGAAAGGGTCCGTCCTTTGCAGACATGGAGG
TCCTGTACTGGACACACGTGAAGGAGCAGTTGGAAACTCTCCGGAAGCTG
CAGAGGAGGGAGGTGGCTGAGCAGTGGCTGCGGCCTGCAGAGGAGGACCA
CCTGGAGGATTCCCTGGAAGACCTGGGGGCAGCAGATGACTTTCTAGAGC
CTGAGGAGTACATGGAGCCCGAGGGAGCAGACCCCAGGGAAGCCGCTGAC
CTTGACGCAGTGCCGATGTCCCTGAGCTACGAGGAGCTGGTTCGAAGGAA
TGTGGAGCTCTTCATCGCCACCTCCCAGAAGTTTGTCCAGGAGACAGAGC
TGAGCCAGCGCATCAGGGACTGGGAGGACACAGTGCAGCCTCTGCTCCAG
GAGCAGGAGCAGCATGTGCCCTTTGACATCCACACCTATGGGGACCAGCT
GGTCTCACGGTTCCCCCAGCTCAATGAGTGGTGTCCCTTTGCGGAGCTGG
TGGCTGGCCAGCCGGCCTTCGAGGTGTGTCGTTCCATGCTGGCCTCCCTG
CAGCTGGCCAATGACTACACAGTGGAGATAACCCAGCAGCCCGGGCTGGA
GATGGCCGTGGACACCATGTCCCTGAGACTGCTCACGCACCAGCGAGCGC
ACAAGCGCTTCCAGACCTACGCTGCCCCCTCCATGGCCCAGCCCTGAGTG
GGGAGCACCGAGGCAGGGGTGGGGGAATGTGTACTGAGGAGCCGTGTGTC
TGCTCCTGGCTGGCCCGGCCTAATAAAGCAGTGTTGCCATCTC

A preferred mutated mouse Nessie polypeptide has the following sequence (SEQ ID NO: 3). The mutation residue is in boldface and the N-terminal cohesin-like domain is underlined:

>mNessie_mutant
MEDVEVRFAHLLQPNRDLTKNWEVDVAAQLGEYLEELDQICISFDEGKTT
MNFIEAALLIQGSACVYSKKVEYLYSLVYQALDFISGKRPAKQLSLVQED
GSKKTVNSETPCETENEFLSLDDFPDSRANVDLKNDQASSELLIIPLLPM
ALVAPDEVEKNSSPLYSCQGDILASRKDFRMNTCMPNPRGCFMLDPVGMC
PVEPVVPVEPYPMSRSQKDPEDAEEQPMEVSRNGSPVPVPDISQEPDGPA
LSGGEEDAEDGAEPLEVALEPAEPRTSQQSAILPRRYMLRERQGAPEPAS
RLQETPDPWQSLDPFDSLESKVFQKGKPYSVPPGVEEAPGQKRKRKGATK
LQDFHKWYLDAYAEHPDGRRARRKGPTFADMEVLYWKHVKEQLETLQKLR
RRKINERWLPGAKQDLWPTEEDRLEESLEDLGVADDFLEPEEYVEEPAGV
MPEEAADLDAEAMPESLRYEELVRRNVELFIATSQKFIQETELSQRIRDW
EDTIQPLLQEQEQHVPFDIHIYGDQLASRFPQLNEWCPFSELVAGQPAFE
VCRSMLASLQLANDYTVEITQQPGLEAAVDTMSLRLLTHQRAHTRFQTYA
APSMAQP

A preferred mutated human Nessie polypeptide has the following sequence (SEQ ID NO: 4). The mutation residue is in boldface and the N-terminal cohesin-like domain is underlined:

>hNessie_mutant
MEDVEARFAHLLQPNRDLTKNWEVDVAAQLGEYLEELDQICISFDEGKTT
MNFIEAALLIQGSACVYSKKVEYLYSLVYQALDFISGKRRAKQLSSVQED
RANGVASSGVPQEAENEFLSLDDFPDSRTNVDLKNDQTPSEVLIIPLLPM
ALVAPDEMEKNNNPLYSRQGEVLASRKDFRMNTCVPHPRGAFMLEPEGMS
PMEPAGVSPMPGTQKDTGRTEEQPMEVSVCRSPVPALGFSQEPGPSPEGP
MPLGGGEDEDAEEAVELPEASAPKAALEPKESRSPQQSAALPRRYMLRER
EGAPEPASCVKETPDPWQSLDPFDSLESKPFKKGRPYSVPPCVEEALGQK
RKRKGAAKLQDFHQWYLAAYADHADSRRLRRKGPSFADMEVLYWTHVKEQ
LETLRKLQRREVAEQWLRPAEEDHLEDSLEDLGAADDFLEPEEYMEPEGA
DPREAADLDAVPMSLSYEELVRRNVELFIATSQKFVQETELSQRIRDWED
TVQPLLQEQEQHVPFDIHTYGDQLVSRFPQLNEWCPFAELVAGQPAFEVC
RSMLASLQLANDYTVEITQQPGLEMAVDTMSLRLLTHQRAHKRFQTYAAP
SMAQP

Wild-type mouse Nessie polypeptide has the following sequence (SEQ ID NO. 5). The N-terminal cohesin-like domain is underlined:

>mNessie_wildtype
MEDVEVRFAHLLQPIRDLTKNWEVDVAAQLGEYLEELDQICISFDEGKTT
MNFIEAALLIQGSACVYSKKVEYLYSLVYQALDFISGKRRAKQLSLVQED
GSKKTVNSETPCETENEFLSLDDFPDSRANVDLKNDQASSELLIIPLLPM
ALVAPDEVEKNSSPLYSCQGDILASRKDFRMNTCMPNPRGCFMLDPVGMC
PVEPVVPVEPYPMSRSQKDPEDAEEQPMEVSRNGSPVPVPDISQEPDGPA
LSGGEEDAEDGAEPLEVALEPAEPRTSQQSAILPRRYMLRERQGAPEPAS
RLQETPDPWQSLDPFDSLESKVFQKGKPYSVPPGVEEAPGQKRKRKGATK
LQDFHKWYLDAYAEHPDGRRARRKGPTFADMEVLYWKHVKEQLETLQKLR
RRKINERWLPGAKQDLWPTEEDRLEESLEDLGVADDFLEPEEYVEEPAGV
MPEEAADLDAEAMPESLRYEELVRRNVELFIATSQKFIQETELSQRIRDW
EDTIQPLLQEQEQHVPFDIHIYGDQLASRFPQLNEWCPFSELVAGQPAFE
VCRSMLASLQLANDYTVEITQQPGLEAAVDTMSLRLLTHQRAHTRFQTYA
APSMAQP

Wild type human Nessie polypeptide has the following sequence (SEQ ID NO: 6). The N-terminal cohesin-like domain is underlined:

>hNessie_wildtype
MEDVEARFAHLLQPIRDLTKNWEVDVAAQLGEYLEELDQICISFDEGKTT
MNFIEAALLIQGSACVYSKKVEYLYSLVYQALDFISGKRRAKQLSSVQED
RANGVASSGVPQEAENEFLSLDDFPDSRTNVDLKNDQTPSEVLIIPLLPM
ALVAPDEMEKNNNPLYSRQGEVLASRKDWFRMNTCVPHPRGAFMLEPEGM
SPMEPAGVSPMPGTQKDTGRTEEQPMEVSVCRSPVPALGFSQEPGPSPEG
PMPLGGGEDEDAEEAVELPEASAPKAALEPKESRSPQQSAALPRRYMLRE
REGAPEPASCVKETPDPWQSLDPFDSLESKPFKKGRPYSVPPCVEEALGQ
KRKRKGAAKLQDFHQWYLAAYADHADSRRLRRKGPSFADMEVLYWTHVKE
QLETLRKLQRREVAEQWLRPAEEDHLEDSLEDLGAADDFLEPEEYMEPEG
ADPREAADLDAVPMSLSYEELVRRNVELFIATSQKFVQETELSQRIRDWE
DTVQPLLQEQEQHVPFDIHTYGDQLVSRFPQLNEWCPFAELVAGQPAFEV
CRSMLASLQLANDYTVEITQQPGLEMAVDTMSLRLLTHQRAHKRFQTYAA
PSMAQP

Wild type mouse Nessie genomic DNA fragments have the following sequence (50 basepairs of intronic flanking sequence is in italics on either side of exon):

GCGCGCAGTGCCCTGGCTACGTACTTCCGGGGCGGGAGCACCAAAATGGC
GCCAGAACCAGTAGCGGGTTGAGCCATGGTTCCGGGCTTCTAGTCCCGTT
CTAGACATGGAGGATGTGGAGGTGCGCTTTGCTCACCTCTTGCAGCCCAT
CCGGGATCTCACTAAGAACTGGGAGGTGGACGTGGCGGCACAGCTGGGCG
AGTATCTGGAGGAGGTGAGGGCGGCGGGGGAATGGCATCGGCCTGGGCGC
TCAGGCTGCATTGC

(SEQ ID NO: 8) exon 2:

AGGCATGCCCTCGCTTACCGAGCCCAGCTTACTAATGTTCTTTCTTGCAG
CCTGGACCAGATCTGCATTTCTTTTGATGAAGGCAAAACCACAATGAACT
TCATTGAGGCAGCACTGTTGATCCAGGGCTCAGCCTGTGTCTACAGTAAG
AAGGTGGGTTCCACAGACCGCCGACAGTTGTGCTTAGTGTTGAGCTCCTG
GTG

(SEQ ID NO: 9) exon 3:

CCCATAGGTCCCTCCTTTCCACATCCTTACTGTGTGTTTCTGGTTGCCAG
GTGGAGTACCTCTACTCGCTGGTCTACCAGGCTCTCGATTTTATTTCTGG
CAAGAGGTGAGTATTAGAAGTGAGCCAGAAGGAAGAGGCGTTTGTTGTCT
TAGCTG

(SEQ ID NO: 10) exon 4:

GGGTCTCTTGAAGCCTCTCAGGATGCTCTTGGTATGTTTTTCTCTCTCAG
GCGGGCCAAACAGCTCTCCTTAGTTCAGGAAGATGGGAGCAAGAAGACTG
TCAACTCAGAGACTCCCTGTGAAACAGAGAATGAGGTTAGTTGGGGCATG
TGAACTCTGCCCTGGGTGCTCGCCTGGCCCTGCTC

(SEQ ID NO: 11) exon 5:

CTGGGTGCTCGCCTGGCCCTGCTCTCATGAGTACTTGTTGTCTCCTGCAG
TTCCTGTCACTGGACGACTTCCCTGACTCCCGGGCTAATGTGGATCTGAA
AAATGATCAGGCATCCAGTGTGAGTGTTCTGGCCCTTGTCCGGTGGGGAG
AGTAGTCTTAGTGCTGTTC

(SEQ ID NO: 12) exon 6:

CTTACTGCTGTTCCGTGTAGAATTCAGAGCTCTCCCCTCCCTACCCACAG
GAGCTGCTTATCATACCCCTACTGCCCATGGCCCTGGTGGCCCCTGATGA
AGTGGAAAAGAACAGCAGCCCCTTGTATAGGTACAACACTCAGCTAGCAT
GGGGGAGAAGGGGAGGACACCTGCCTGGGA

(SEQ ID NO: 13) exon 7:

ACACCTGCCTGGGAAGGGCGTGCGTAGCAAGTCTGCTTTTGCCAACACAG
CTGTCAGGGTGACATCTTGGCCAGCCGGAAGGATTTCAGGATGAACACGT
GTATGCCTAACCCCAGAGGCTGCTTTATGTTAGATCCAGTGGGAATGTGT
CCTGTGGAGCCTGTGGTGCCCGTGGAGCCATACCCCATGTCAAGGAGCCA
GAAAGGTAAGGGTTTGGATGTGGGAGCTTAGTGGGAAGGAAGTAGTATCA
GTCTC

(SEQ ID NO: 14) exon 8:

GAACCCTTGTAGGGGCGGAGCTGACTCTGTCTAGTGTCTGTCTCTCCTAG
ATCCTGAGGACGCTGAGGAGCAGCCCATGGAAGTGTCTAGGAACGGGAGT
CCTGTTCCTGTACCCGACATCTCCCAAGAGCCAGGTGAGAAGAGAGTACC
AGGGAGACTGAGCTGGGTGGACTCCGACCCTGGA

(SEQ ID NO: 15) exon 9:

GCCTCTTTGGGCTAACTGGTTTCTTGGTTCCTGCACCAACATTTTTCTAG
ATGGTCCAGCGCTCAGCGGTGGAGAGGAGGATGCAGAGGATGGAGCAGAG
CCCCTGGAGGTTGCTCTAGAGCCTGCAGAGCCAAGGACCTCACAGCAGGT
GGGACACCAATGAGGCTGCAGAGCCCAAGCTGTCAACAGAGTAAGCCC

(SEQ ID NO: 16) exon 10:

CTGGGTGGACAGGGGTTGGAAGAGGAGAACCTGTGGGTTGTTTCTTGCAG
AGTGCCATCTTGCCAAGGAGATACATGCTGCGGGAACGACAAGGGGCACC
GGAGCCTGCCTCCCGGCTACAGGTGAGAAATGTGAGGTCCCTAGACCCTT
CAGACCCTTGGCACGTCTTCCA

(SEQ ID NO: 17) exon 11:

GAGACCTAATGCTTTCCCCTTCTCAGCTTCTACTTGTGTTTCCTGCTCAG
GAGACCCCAGACCCCTGGCAGAGCCTGGACCCTTTTGACTCCTTGGAATC
TAAGGTCTTCCAGAAAGGTAAATAGGATTGGTGACACCTTGCTCGAGTTG
CCTTGCCCAGGGCTCCT

(SEQ ID NO: 18) exon 12:

TGGATCGCTTTCCCTTGGCTACTCTTGAGGCAGTCCCTCTCTGCCTCCAG
GGAAACCCTATTCTGTGCCACCCGGTGTGGAGGAGGCTCCAGGACAGAAG
CGCAAGAGGAAGGGTGCCACCAAGTTGCAGGACTTCCACAAGTGGTACCT
GGATGCCTGTGAGTGAGTATATGGGCTGAGGTGACACACCCGTTTTCCTG
GGTGTTGG

(SEQ ID NO: 19) exon 13:

CATCGGGCACTGGGGTTTCGGCCTGGCGCTTGCTAACTACTTTCTCCCAG
ATGCTGAACACCCTGACGGCAGGAGGGCTCGGCGGAAGGGCCCAACCTTT
GCAGGTGAGGCTAGGGTCCTATGTCCATCCTCAGACAACAGTCTTTCTCT
TCCT

(SEQ ID NO: 20) exon 14:

GTGAGCTTGGGCCGGATGGGGAAATCCCCTTATAAGGTCTTTGTTTGCAG
ACATGGAAGTCCTGTACTGGAAACATGTGAAAGAACAGCTCGAGACCCTT
CAGAAGCTGCGGAGACGTAAGGCAAGTACCCACAGGACCAAGGTCTGCAA
CCCCACAGGTCTGAGGCATGG

(SEQ ID NO: 21) exon 15:

GGTCTGAGGCATGGGATATATGAGTTTCCTTACAGATGTTCTCTGGGCAG
ATCAACGAGAGATGGCTACCTGGGGCCAAGCAGGATCTGTGGCCTACAGA
GGAGGATCGCTTGGAAGAGTCCCTCGAGGACCTCGGGGTAGCAGGTGGGT
GCCTTTAGATGGAATGTGCGTGTGTTGTGTGGTCAGAGTCGTGC

(SEQ ID NO: 22) exon 16:

GTACTGCTGGCTGAGCAGCAGCCTTGCTCTCTTCTCCCATGTCTCAGCAG
ATGACTTTCTAGAGCCCGAGGAGTACGTGGAGGAGCCTGCGGGGGTGATG
CCCGAGGAAGCTGCTGACCTCGGTAGGTCTGTGATGGGGAGCGGCAGGAG
GGGAGAATGTAGCCCCAGAGCT

(SEQ ID NO: 23) exon 17:

GGGAGAATGTAGCCCCAGAGCTCTAGCAGCTCATGGTTGGACCTTTTTAG
ATGCAGAGGCCATGCCAGAGTCCCTGAGATACGAGGAGCTGGTCCGGAGA
AATGTGGTAGGCCTGGGTCACCAAGGTGGGAGCAGTGGGGACATCCTGGG
CCCATG

(SEQ ID NO: 24) exon 18:

GACATCCTGGCCCCATGTGTGCAAGGGAGAACCCACTGGTCCTTCTGTAG
GAACTCTTCATTGCCACCTCCCAGAAGTTTATCCAGGAGACAGAGCTGAG
CCAACGCATCAGGGACTGGGAAGATACCATCCAGCCCCTGCTCCAGGAGC
AGGTGAGGCGTGCTATTGGGGACTAGAGCCTCCTCCCACCAGCCCGGGGT
TT

(SEQ ID NO: 25) exon 19:

GCCCAGACCTGTCTCTCCACTTGAAGCTAACATGTCCTTTCCCTCTGCAG
GAGCAGCATGTGCCCTTTGATATCCATATCTACGGGGACCAGTTGGCTTC
ACGGTTCCCCCAGCTCAATGAATGGTGTCCCTTTTCAGAGCTTGTAGCAG
GGCAGCCTGCTTTTGAGGTGTGCCGCTCCATGCTGGCCTCCTTGCAACTG
GTAAGTGGCCTGGGACACAAGGGATGGGGCAGCGGCCCTGGACTCTACTG

(SEQ ID NO: 26) exon 20:

CAAGGGATGGGGCAGCGGCCCTGGACTCTACTGACACCTTGTTTCCACAG
GCTAATGACTACACAGTGGGAGATCACTCAGCAGCCAGGACTGGAGGCAG
CTGTGGACACAATGTCTCTGAGACTGCTCACACACCAGCGAGCCCACACC
CGCTTCCAGACCTATGCTGCACCATCCATGGCCCAGCCTTGAGTGGACAG
CACTGAGGCAGGGGTGGAAAGTAGTATATACCTGGAGGTCTTTGCCCCTA
ATGTGCTATGGGGCCATTCACTCCAGTGCTGCCTCCTGGCTGGCCTAGCC
TAATAAAGTGTTGCTACCCCACCTGTTCACCGGACAGACTATTTAAATGA
GCTGC

A preferred wildtype mouse Nessie genomic DNA subfragment has the following sequence (SEQ ID NO: 27):

TTTGCTCACCTCTTGCAGCCCATCCGGGATCTCACTAAGAACTGGG

A preferred mutated mouse Nessie genomic DNA subfragment has the following sequence (SEQ ID NO: 28) (the mutation site, relative to mouse wildtype Nessie is in bold underline):

TTTGCTCACCTCTTGCAGCCCAACCGGGATCTCACTAAGAACTGGG

Wild type human Nessie genomic DNA fragments have the following sequence (50 basepairs of intronic flanking sequence is in italics on either side of exon):

(SEQ ID NO: 29) exon 1:

CGACGCCGCGCCTACGCATTTTCCTGGGCGGGAACAGCAAAATGGCGCCA
GAACTAGTGGCGGGCTGAGGACGCCGTACCCCTCGGAAGGCAGCCCTGCG
GTCCCTTTGCCGCCCGTTCCCTCCCGGACATGGAGGACGTGGAGGCGCGC
TTCGCCCACCTCTTGCAGCCCATCCGCGACCTCACCAAGAACTGGGAGGT
GGACGTGGCGGCCCAGCTGGGCGAGTATCTGGAGGAGGTAAGGGCGGCGG
GGGAGTGACGCCGGGTGGGCCGGCGGGTGGGGCTCCG

(SEQ ID NO: 30) exon 2:

AGTGCAGACTGGGCCTTGGATTCCCCCTGTCTTTGTGTTGTTTCTTGCAG
CTGGATCAGATCTGCATTTCTTTTGACGAAGGCAAGACCACAATGAACTT
CATTGAGGCAGCGTTGTTGATCCAGGGCTCTGCCTGCGTCTACAGTAAGA
AGGTGGGCCCTGCTTGACGCTGGTCTTGGCATTTTGGTGGCCAGTGGGAC
CA

(SEQ ID NO: 31) exon 3:

GGGGTGGGCCCCTCCTTTCTGGGTCCTCACTGCCTGCATCTGGTCACCAG
GTGGAATACCTCTACTCACTCGTCTACCAGGCCCTTGATTTCATCTCTGG
AAAGAGGTGAGTTCTGCAGCCACTCACTGTGCTGCCCTGCATGTCGCCAG
GGAGGC

(SEQ ID NO: 32) exon 4:

GGAGGCCCCTGCAGCTCCTGGGATGCCCACGGGATGTGCTTCTCTCTCAG
GCGGGCCAAGCAGCTCTCTTCGGTGCAGGAGGACAGGGCCAATGGGGTTG
CCAGCTCCGGGGTCCCCCAGGAGGCAGAGAATGAGGTGAGTTTCTTTGGC
ATGTGGTCCCCGCCCACTGTGTGTTTGCCTGGGCT

(SEQ ID NO: 33) exon 5:

CACTGTCTGTTTGCCTGGGCTCCGCCCCCACGAGCTCTGTCTCCCTCCAG
TTCCTGTCGCTGGATGACTTCCCTGACTCCCGGACTAACGTGGATCTCAA
GAATGATCAGACGCCCAGTGTGAGTCCTGGCCTGGCCCCTCTTAGGCTGG
GGTGAGGTCAGCACTTTCC

(SEQ ID NO: 34) exon 6:

CCACTGGAGTGGAAGGGTGCCTGGCTCACCCACCCTTGGCCTCCATGCAG
GAGGTCCTCATCATCCCCCTCCTGCCCATGGCCCTGGTGGCCCCTGATGA
AATGGAGAAGAACAACAATCCCCTGTACAGGTAGGGATCTGAGCCCAGCG
ACGGGGAGGGAGGCCTGCCTGGGAAGGGTC

(SEQ ID NO: 35) exon 7:

AGGCCTGCCTGGGAAGGGTCTCTACAGCAGGCGTGTTTTTGCCAGCACAG
CCGTCAGGGTGAGGTCCTGGCCAGCCGGAAGGATTTCAGGATGAACACGT
GCGTTCCCCACCCCAGAGGGGCCTTCATGTTGGAGCCAGAGGGCATGTCC
CCCATGGAACCAGCGGGCGTTTCCCCCATGCCAGGGACCCAGAAGGGTGA
GGGCTTGGATGCGGGGGGCTTGGTGGGAAGGAAGGGAGGGTCTTCT

(SEQ ID NO: 36) exon 8:

CATCTTGGAGAGGGGCTGGGCTGACCTTGTCTGATCCCTGTCTCTCCCAG
ACACCGGGAGGACTGAGGAGCAGCCAATGGAAGTTTCCGTGTGCAGGAGC
CCTGTCCCAGCACTCGGCTTCTCCCAGGAGCCAGGTGAGAAGAGAGCTCC
CCGGTGGGACTGGCAGGGCAGCCAAAGAGGGGAC

(SEQ ID NO: 37) exon 9:

TCTGGTCTCGGCACTCCTTGGAGCTGATCACTCTCTTGCTCCCTGCCTAG
GCCCCTCTCCAGAAGGCCCGATGCCCCTGGGTGGGGGCGAGGACGAGGAT
GCAGAGGAGGCAGTAGAGCTTCCTGAGGCCTCGGCCCCCAAGGCCGCTCT
GGAGCCCAAGGAGTCCAGGAGCCCGCAGCAGGTGGGACCCACATGGAGGC
CTGCAGAACCTGAGCTGTGAACTGGCAACCC

(SEQ ID NO: 38) exon 10:

CCTTGAGGGGAGAAAGAGGAGAAGTGCGGACCCTGTGACTGTCTTTGCAG
AGTGCTGCCCTGCCCAGGAGGTACATGCTGCGGGAGCGGGAGGGGGCCCC
AGAGCCTGCATCCTGCGTGAAGGTAGGAGTGTTGGGGCCCTGACCCCCGG
CAGGGAGGGATGGGCGGATTCG

(SEQ ID NO: 39) exon 11:

CGGCTGTGCACCCCCTACTTCTCCAGCGCCTTCTTGTGCTCTGTGCCCAG
GAGACTCCAGACCCCTGGCAGAGCCTGGACCCCTTTGACTCCTTGGAGTC
TAAGCCCTTCAAGAAAGGTAATTGGGTGGAAGGTACCTCCACTCAGGTAC
CCCTGGCTGCGTTTCCT

(SEQ ID NO: 40) exon 12:

TTGCACCCTGATCCCCCAGCGGCTCTAAGACAGTCCCTGTTTGCCCCCAG
GTAGGCCTTACTCTGTGCCCCCCTGTGTGGAGGAGGCTCTGGGACAGAAG
CGCAAGAGGAAGGGCGCTGCCAAGCTGCAGGACTTCCACCAGTGGTACCT
GGCTGCCTGTGAGTGGGTGTGGTGTGCACTCCGGACCACTGGGAGCTGGG
GGCTGGGC

(SEQ ID NO: 41) exon 13:

TGCTGTGGGCAGCTCTTGGCCTGGCTGGTGCTGAGCATGTTCTTTCACAG
ATGCAGACCATGCCGACAGCAGGCGGCTTCGGCGAAAGGGTCCGTCCTTT
GCAGGTGAGGCTGAAGTCCTCGGGGAAGACAGTTTTACTCTCCTTCCCCT
ACCT

(SEQ ID NO: 42) exon 14:

CACGGGGCCTGGGAAGGACGAGGGAGCCCTCACAAGGCCTTTGTCTGCAG
ACATGGAGGTCCTGTACTGGACACACGTGAAGGAGCAGTTGGAAACTCTC
CGGAAGCTGCAGAGGAGGGAGGCAAGTCCCAGCTGGTCAGCTGTGATCTA
GGACCCCGTGGTGGCCCTGAT

(SEQ ID NO: 43) exon 15:

GGCCCTGATGGGAGGTGACAGTGCCCCTCACAGATGCTTTCTCTGGACAG
GTGGCTGAGCAGTGGCTGCGGCCTGCAGAGGAGGACCACCTGGAGGATTC
CCTGGAAGACCTGGGGGCAGCAGGTGGGTGCCTGCCAGGGGGTGGGGTGG
GGCTTGGCACCTGCCGACTAGCT

(SEQ ID NO: 44) exon 16:

ACCTGCCGACTAGCTGCCTGCCTGCTGTTCACTCTCCCACCTCCCAGCAG
ATGACTTTCTAGAGCCTGAGGAGTACATGGAGCCCGAGGGAGCAGACCCC
AGGGAAGCCGCTGACCTTGGTAGGTGGGCAGCGGGCTAGGAGTGCTGAGG
GGCCACTGGAGCTGGGGGC

(SEQ ID NO: 45) exon 17:

GGGGAGAGCGTGGCCCCTTAGCTGCCCAGCTCACAGCTACCCCTTCCCAG
ACGCAGTGCCGATGTCCCTGAGCTACGAGGAGCTGGTTCGAAGGAATGTG
GTAGGCCTGGGTTAGAGGGAGACGGGGAGGGGAGGGGGACAGGTGAGCGG

(SEQ ID NO: 46) exon 18:

TGCCCAGGCCCCTGCTTGGGAGGCAGTAGCTCCTGCTGATCCTCCCCTAG
GAGCTCTTCATCGCCACCTCCCAGAAGTTTGTCCAGGAGACAGAGCTGAG
CCAGCGCATCAGGGACTGGGAGGACACAGTGCAGCCTCTGCTCCAGGAGC
AGGTGAGGCGGGGCCGCTGGGAACCAGAGCTGTGTGCCACGGGTCTGTCC
AG

(SEQ ID NO: 47) exon 19:

GTCCAGGGCCTTGCCTCTCTCCGCAGCCAACATGCCCCTCCCCTGTGCAG
GAGCAGCATGTGCCCTTTGACATCCACACCTATGGGGACCAGCTGGTCTC
ACGGTTCCCCCAGCTCAATGAGTGGTGTCCCTTTGCGGAGCTGGTGGCTG
GCCAGCCGGCCTTCGAGGTGTGTCGTTCCATGCTGGCCTCCCTGCAGCTG
GTGAGTAGCCTGGGATACGTGGGAGGGGGAGACGGTCCCCAGACCCTGCT

(SEQ ID NO: 48) exon 20:

GTGGGAGGGGGAGACGGTCCCCAGACCCTGCTGATGTGCCACCCCTGCAG
GCCAATGACTACACAGTGGAGATAACCCAGCAGCCCGGGCTGGAGATGGC
CGTGGACACCATGTCCCTGAGACTGCTCACGCACCAGCGAGCGCACAAGC
GCTTCCAGACCTACGCTGCCCCCTCCATGGCCCAGCCCTGAGTGGGGAGC
ACCGAGGCAGGGGTGGGGGAATGTGTACTGAGGAGCCGTGTGTCTGCTCC
TGGCTGGCCCGGCCTAATAAAGCAGTGTTGCCATCTCATCTTCCCCCTAA
AAACCCTTTTATGTACACCTGCGCAGAGAAGAGGGCT

A preferred wildtype human Nessie genomic DNA subfragment has the following sequence (SEQ ID NO: 49):

TTCGCCCACCTCTTGCAGCCCATCCGCGACCTCACCAAGAACTGGG

A preferred mutant human Nessie genomic DNA subfragment has the following sequence (SEQ ID NO: 50) (the mutation site, relative to mouse wildtype Nessie is in bold underline):

TTCGCCCACCTCTTGCAGCCCAACCGCGACCTCACCAAGAACTGGG

Identification and Characterization of Novel Nessie Sequences

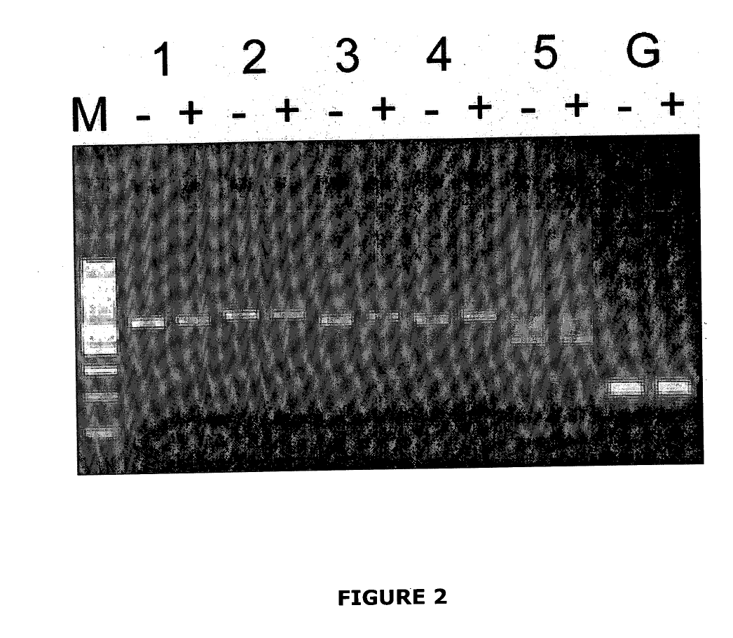

The Nessie gene was initially identified in the mouse genome on chromosome 15. A 1993 basepair mRNA sequence with a 1821 basepair coding sequence translates into a 607 amino acid protein. The mouse gene is comprised of at least 20 exons spanning approximately 15.6 kilobases of sequence (see FIG. 1). ESTs imply that the Nessie gene is expressed during different developmental time points and in different tissues. The expression of the Nessie gene in the mouse thymus was documented by RT-PCR (see FIG. 2).

Identification of Nessie orthologs in other species can be performed using a number of methods known to those of skill in the art. These methods include computational genomic annotation, using programs such as BLAST and GeneScan (see, e.g., Lynn et al., J. Genet. 80: 9-16 (2001)); and biochemical methods, e.g., low stringency hybridization methods. For computational identification, BLAST version 2.0 is preferably used with parameters set at word size=3, expect=10, filter low complexity with SEG, cost to open gap=11, cost to extend gap=1, similarity matrix Blosum62, Dropoff (X) for blast extensions in bits=7, X dropoff value for gapped alignment (in bits)=15, final X dropoff value for gapped alignment=25.

In addition, one may isolate the genes encoding the novel polypeptides using methods known to the skilled artisan. For example, cDNA encoding a protein of interest may be identified by screening a cDNA library that can be obtained as an EcoRI-based lambda phage library (lambda ZAP) from Stratagene Cloning Systems (La Jolla, Calif., U.S.A.). The cDNA library may be screened, for example, using a plurality of random oligonucleotide probes constructed based on the known amino acid sequence obtained from a protein of interest using mass spectrometry. Exemplary conditions for screening comprise 6 times SSC, 25% formamide, 5% Dernhardt's solution, 0.5% SDS, 100 μg/ml denatured salmon sperm DNA, at 42° C. Exemplary processing of such screens comprise the following steps: filters are washed with 2 times SSC containing 0.5% SDS at 25° C. for 5 minutes, followed by a 15 minute wash at 50° C. with 2 times SSC containing 0.5% SDS; the final wash is with 1 times SSC containing 0.5% SDS at 50° C. for 15 minutes; filters are exposed to X-ray film (Kodak) overnight; from positive clones screened, cDNA inserts are identified. The sequences of identified cDNAs may be determined, for example, by purifying plaques containing the cDNAs identified, and excising as phagemids according to the supplier's specifications, to generate insert-carrying Bluescript-SK variants of the phagemid vectors. Sequencing of the relevant clones across their entire sequence should reveal a putative ATG initiation codon together with an oligonucleotide of 5′ non-coding region and the coding region having a polyA splice site.

Once Nessie nucleic acids and/or polypeptides have been identified, mutations in these sequences can be readily identified by comparison to these “normal” sequences. DNA and polypeptide sequencing methods are well known to those of skill in the art. As noted above, a mutated Nessie polypeptide refers to a polypeptide that exhibits a deficiency in one or more characteristics displayed by “normal” Nessie proteins, such as an amino acid substitution, deletion, or insertion. Preferred mutated Nessie polypeptides exhibit a detectable phenotypic effect when expressed in cells or animals. Particularly preferred mutated Nessie polypeptides produce a recessive altered immune system-related phenotype in an animal.

Mutations generated spontaneously may be identified by phenotypic effects that are known to relate to Nessie function (e.g., immune system-related phenotypes seen in the Nessie mutation), and confirmed by direct sequencing of the putative mutant; by chromosome mapping; or by a combination of these methods. As an alternative to identifying spontaneous mutations in Nessie sequences, methods for introducing mutations into a known “normal” Nessie sequence can be performed. Such methods include site specific mutagenesis, in which a change in a nucleic acid sequence is introduced at a predetermined location in the sequence, and the nucleic acid is then introduced into a cell; gene transfer methods, in which a gene transfer vector is used to introduce a desired nucleic acid sequence into a cell genome; and “knockout” and “knockin” methods, in which a nucleic acid sequence is introduced into the genome of a cell at a specific location, often substituting an introduced gene for a genomic version (see, e.g., Kuhn, Science 269: 1427-9 (1995)).

In preferred embodiments, mutations may be induced by exposing a cell or whole animal to one or more mutagens, which randomly introduce mutations into the cell or animal genome. Suitable mutagens include, but are not limited to, radiation (gamma, beta, alpha, UV, etc.); base analogues such as bromouracil and aminopurine; chemicals such as nitrous acid, nitrosoguanidine, ethylnitrosourea (“ENU”), and ethylmethanesulfonate; intercalating agents such as acridine orange and ethidium bromide, to provide endogenous mutated Nessie nucleic acid sequences. ENU is preferred for use in whole animal, most preferably whole mouse, mutagenesis. See, e.g., Rinchik, Trends Genet. 7: 15-21 (1991). Protocols are available which provide a very efficient mutagenesis rate in mice. See, e.g., Favor, et al., Mut. Res. 231: 47-54 (1990).

The mutations induced in these manners may include deletions, substitutions, inversions, insertions, etc., however, the mutations recovered after ENU mutagenesis are mainly point mutations. Many of the mutants produced by ENU will therefore be hypomorphic (partial loss-of-function) mutations, although also gain-of-function as well as complete loss of function mutants can be expected. The frequency of mutant recovery from ENU methods is about 1/1000 for a specific locus that can be scored phenotypically, but strain, dosage and treatment regimen do influence the mutagenesis rate.

Methods for introducing or inducing mutations can be advantageously combined with nuclear transfer cloning methods to provide an animal comprising a mutated sequence of interest, e.g., by transferring the genetic material from a cell harboring the mutated sequence, preferably an embryonic stem cell in mice, into an enucleated oocyte; or with methods of blastocyst injection of genetically altered embryonic stem cells to provide germline chimeras; or with methods of pronuclear injection of fertilized mouse eggs with DNA constructs. See, e.g., Rideout et al., Cell 109: 17-27 (2002); Rideout et al., Nat. Genet. 24: 109-10 (2000); Nakao et al., Exp. Anim. 47:167-71 (1998). Alternatively, ENU can be used to mutagenize premeiotic spermatogonial stem cells in male animals, allowing the production of a large number of F1 founder animals from a single treated male.

Both dominant and recessive screens can be used to characterize mutations. Typically, male mice can be injected with ENU and then mated to females in order to produce G1 founders. These G1 mice can either be analyzed directly for dominant mutations or bred further to subsequently study recessive phenotypes. Very large numbers of mice can be analyzed in a dominant G1 screen. In this case, all G1 mice are screened for phenotypic abnormalities.

The screen for recessive mutations will involve at least two generations of breeding. From G1 founder males, G2 female offspring are raised, half of which are heterozygous for the newly induced mutations. Backcrossing G2 to the G1 founder male or intercrossing the G2 offspring is then carried out to provide homozygous G3 offspring. Recessive mutant phenotypes are then identified among the G3 offspring.

Manipulation of Novel Nessie Sequences

Techniques for the manipulation of nucleic acids, such as, e.g., subcloning, labeling probes (e.g., random-primer labeling using Klenow polymerase, nick translation, amplification), sequencing, hybridization and the like are well disclosed in the scientific and patent literature, see, e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, Tijssen, ed. Elsevier, N.Y. (1993).

Nucleic acid sequences can be amplified as necessary for further use using amplification methods, such as PCR, isothermal methods, rolling circle methods, etc., are well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun. 1; 29(11): E54-E54; Hafner et al., Biotechniques 2001 April; 30(4):852-6, 858, 860 passim.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be performed by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The nucleic acids of the invention can also be provided in expression vectors and cloning vehicles, e.g., sequences encoding the polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are disclosed, e.g., U.S. Pat. No. 5,426,039. To facilitate cloning of amplified sequences, restriction enzyme sites can be “built into” a PCR primer pair. Vectors may be introduced into a genome or into the cytoplasm or a nucleus of a cell and expressed by a variety of conventional techniques, well described in the scientific and patent literature. See, e.g., Roberts (1987) Nature 328:731; Schneider (1995) Protein Expr. Purif. 6435:10; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous merits of each of the parent vector properties (See e.g., Feng (1997) Nature Biotechnology 15:866-870). Such viral genomes may be modified by recombinant DNA techniques to include the nucleic acids of the invention; and may be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof. See, e.g., U.S. Pat. Nos. 6,117,681; 6,107,478; 5,658,775; 5,449,614; Buchscher (1992) J. Virol. 66:2731-2739; Johann (1992) J. Virol. 66:1635-1640). Adeno-associated virus (AAV)-based vectors can be used to transduce cells with target nucleic acids, e.g., in the in vitro production of nucleic acids and peptides, and in in vivo and ex vivo gene therapy procedures. See, e.g., U.S. Pat. Nos. 6,110,456; 5,474,935; Okada (1996) Gene Ther. 3:957-964.

The present invention also relates to fusion proteins, and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well disclosed in the scientific and patent literature, see e.g., Kroll (1993) DNA Cell. Biol., 12:441-53.

The nucleic acids and polypeptides of the invention can be bound to a solid support, e.g., for use in screening and diagnostic methods as described hereinafter. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of molecules to a solid support can be direct (i.e., the molecule contacts the solid support) or indirect (a “linker” is bound to the support and the molecule of interest binds to this linker). Molecules can be immobilized either covalently (e.g., utilizing single reactive thiol groups of cysteine residues (see, e.g., Colliuod (1993) Bioconjugate Chem. 4:528-536) or non-covalently but specifically (e.g., via immobilized antibodies (see, e.g., Schuhmann (1991) Adv. Mater. 3:388-391; Lu (1995) Anal. Chem. 67:83-87; the biotin/streptavidin system (see, e.g., Iwane (1997) Biophys. Biochem. Res. Comm. 230:76-80); metal chelating, e.g., Langmuir-Blodgett films (see, e.g., Ng (1995) Langmuir 11:4048-55); metal-chelating self-assembled monolayers (see, e.g., Sigal (1996) Anal. Chem. 68:490-497) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce Chemicals, Rockford, Ill.).

Antibodies can also be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon (1989) Nature 377:525-531 (1989).

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface. In practicing the methods of the invention, any known array and/or method of making and using arrays can be incorporated in whole or in part, or variations thereof, as disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Host Cells and Transformed Cells Comprising Novel Nessie Sequences

The invention also provides a transformed cell comprising a nucleic acid sequence of the invention, e.g., a sequence encoding a polypeptide of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

Vectors may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

For transient expression in mammalian cells, cDNA encoding a polypeptide of interest may be incorporated into a mammalian expression vector, e.g. pcDNA1, which is available commercially from Invitrogen Corporation (San Diego, Calif., U.S.A.; catalogue number V490-20). This is a multifunctional 4.2 kb plasmid vector designed for cDNA expression in eukaryotic systems, and cDNA analysis in prokaryotes; incorporated on the vector are the CMV promoter and enhancer, splice segment and polyadenylation signal, an SV40 and Polyoma virus origin of replication, and M13 origin to rescue single strand DNA for sequencing and mutagenesis, Sp6 and T7 RNA promoters for the production of sense and anti-sense RNA transcripts and a Col E1-like high copy plasmid origin. A polylinker is located appropriately downstream of the CMV promoter (and 3′ of the T7 promoter).

The cDNA insert may be first released from the above phagemid incorporated at appropriate restriction sites in the pcDNAI polylinker. Sequencing across the junctions may be performed to confirm proper insert orientation in pcDNAI. The resulting plasmid may then be introduced for transient expression into a selected mammalian cell host, for example, the monkey-derived, fibroblast like cells of the COS-1 lineage (available from the American Type Culture Collection, Rockville, Md. as ATCC CRL 1650).

For transient expression of the protein-encoding DNA, for example, COS-1 cells may be transfected with approximately 8 μg DNA per 106 COS cells, by DEAE-mediated DNA transfection and treated with chloroquine according to the procedures described by Sambrook et al, Molecular Cloning: A Laboratory Manual, 1989, Cold Spring Harbor Laboratory Press, Cold Spring Harbor N.Y., pp. 16.30-16.37. An exemplary method is as follows. Briefly, COS-1 cells are plated at a density of 5×106 cells/dish and then grown for 24 hours in FBS-supplemented DMEM/F12 medium. Medium is then removed and cells are washed in PBS and then in medium. A transfection solution containing DEAE dextran (0.4 mg/ml), 100 μM chloroquine, 10% NuSerum, DNA (0.4 mg/ml) in DMEM/F12 medium is then applied on the cells 10 ml volume. After incubation for 3 hours at 37° C., cells are washed in PBS and medium as just described and then shocked for 1 minute with 10% DMSO in DMEM/F12 medium. Cells are allowed to grow for 2-3 days in 10% FBS-supplemented medium, and at the end of incubation dishes are placed on ice, washed with ice cold PBS and then removed by scraping. Cells are then harvested by centrifugation at 1000 rpm for 10 minutes and the cellular pellet is frozen in liquid nitrogen, for subsequent use in protein expression. Northern blot analysis of a thawed aliquot of frozen cells may be used to confirm expression of receptor-encoding cDNA in cells under storage.

In a like manner, stably transfected cell lines can also prepared, for example, using two different cell types as host: CHO KI and CHO Pro5. To construct these cell lines, cDNA coding for the relevant protein may be incorporated into the mammalian expression vector pRC/CMV (Invitrogen), which enables stable expression. Insertion at this site places the cDNA under the expression control of the cytomegalovirus promoter and upstream of the polyadenylation site and terminator of the bovine growth hormone gene, and into a vector background comprising the neomycin resistance gene (driven by the SV40 early promoter) as selectable marker.

An exemplary protocol to introduce plasmids constructed as described above is as follows. The host CHO cells are first seeded at a density of 5×10⁵ in 10% FBS-supplemented MEM medium. After growth for 24 hours, fresh medium is added to the plates and three hours later, the cells are transfected using the calcium phosphate-DNA co-precipitation procedure (Sambrook et al, supra). Briefly, 3 μg of DNA is mixed and incubated with buffered calcium solution for 10 minutes at room temperature. An equal volume of buffered phosphate solution is added and the suspension is incubated for 15 minutes at room temperature. Next, the incubated suspension is applied to the cells for 4 hours, removed and cells were shocked with medium containing 15% glycerol. Three minutes later, cells are washed with medium and incubated for 24 hours at normal growth conditions. Cells resistant to neomycin are selected in 10% FBS-supplemented alpha-MEM medium containing G418 (1 mg/ml). Individual colonies of G418-resistant cells are isolated about 2-3 weeks later, clonally selected and then propagated for assay purposes.

Nessie Binding Proteins and Substrates

The present invention provides methods for identifying polypeptides that bind to one or more Nessie polypeptides, typically through use of host cells expressing Nessie. These methods may include in vitro or in vivo characterization of proteins that by binding Nessie may modulate or be modulated by Nessie activity or expression levels.

Such identification methods include the yeast two-hybrid system in which Nessie full-length polypeptide or Nessie fragments including the N-terminal cohesin-like domain are expressed in yeast as “bait” fusion proteins in a screen against a cDNA library of “prey” fusion proteins. The fusion components of the screening system are typically the transactivation domain and DNA binding domain of a transcription factor such as yeast GAL4. When bait and prey bind each other, GAL4 transcriptional activation activity is reconstituted, upregulating transcription of a reporter gene construct. Such reporter constructs can be composed of GAL4 DNA binding sites upstream of a minimal promoter and marker gene such as lacZ, and library clones with increased reporter gene activity are identified by staining with β-D-galactoside.

Binding proteins can also be identified by applying biochemical approaches to host cells expressing Nessie. In one embodiment, purified fusion protein consisting of Nessie polypeptides of the invention and a tag such as FLAG is incubated in vitro with lysate from a cell type or cell line of interest or candidate or test substrates under conditions that allow protein binding. Using antibodies against the FLAG tag, Nessie can then be pulled down together with any binding proteins and separated by protein gel electrophoresis or by other means such as mass spectrophotometry. Peptide microsequencing of the binding protein band can identify the binding protein by amino acid sequence.

Screening Methodologies

The present invention also provides methods for identifying compositions that affect one or more biochemical characteristics of Nessie polypeptides and/or mutated Nessie polypeptides, and/or that affect, and preferably ameliorate, an aberrant phenotype displayed by a cell, tissue, organ, or animal expressing Nessie polypeptides and/or mutated Nessie polypeptides.

These screening methods include target-based screening methods (i.e., screening of compositions for their activity at a protein or nucleic acid in vitro), cell-based screening methods, and whole animal-based screening methods. In practicing the methods of the invention, a variety of apparatus and methodologies can be used to in conjunction with the polypeptides and nucleic acids of the invention, e.g., to screen compositions that act as potential modulators of one or more of these characteristics. A “modulator” of the invention denotes compounds that alter the activity of another compound, enzyme, protein, nucleic acid or receptor in comparison to a control or the expected activity of the other compound, enzyme, protein, nucleic acid or receptor. Thus the term embraces both inhibitors, agonists, antagonists, partial agonists and other compounds which increase or decrease activity of the other compound, enzyme, protein, nucleic acid or receptor. Modulators may act directly or indirectly by, e.g., competitive or non-competitive binding to a receptor or enzyme, by increasing or decreasing protein levels such as by a targeted genetic disruption, increasing or reducing transcription of a gene, increasing protein instability, and the like.

In practicing the screening methods of the invention, a test compound can be contacted with a polypeptide of the invention in vitro or administered to a cell of the invention or an animal of the invention in vivo. Combinatorial chemical libraries are one means to assist in the generation of new chemical leads compounds.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds (see, e.g., Gallop et al. (1994) 37(9): 1233-1250). Preparation and screening of combinatorial chemical libraries are well known to those of skill in the art, see, e.g., U.S. Pat. Nos. 6,004,617; 5,985,356. Such combinatorial chemical libraries include, but are not limited to, peptide libraries (see, e.g., U.S. Pat. No. 5,010,175; Furka (1991) Int. J. Pept. Prot. Res., 37: 487-493, Houghton et al. (1991) Nature, 354: 84-88). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs (1993) Proc. Nat. Acad. Sci. USA 90: 6909-6913), vinylogous polypeptides (see, e.g., Hagihara (1992) J. Amer. Chem. Soc. 114: 6568), non-peptidal peptidomimetics with a Beta-D-Glucose scaffolding (see, e.g., Hirschmann (1992) J. Amer. Chem. Soc. 114: 9217-9218), analogous organic syntheses of small compound libraries (see, e.g., Chen (1994) J. Amer. Chem. Soc. 116: 2661), oligocarbamates (see, e.g., Cho (1993) Science 261:1303), and/or peptidyl phosphonates (see, e.g., Campbell (1994) J. Org. Chem. 59: 658). See also Gordon (1994) J. Med. Chem. 37:1385; for nucleic acid libraries, peptide nucleic acid libraries, see, e.g., U.S. Pat. No. 5,539,083; for antibody libraries, see, e.g., Vaughn (1996) Nature Biotechnology 14:309-314; for carbohydrate libraries, see, e.g., Liang et al. (1996) Science 274: 1520-1522, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. Nos. 6,045,755; 5,792,431; 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Once suitable lead compounds have been identified, rational drug design methods can be used to optimize the utility of the compound as a biopharmaceutical agent. In these methods, 3-dimensional structure information obtained from x-ray crystallographic or NMR studies of a target polypeptide is used to specifically produce or modify a therapeutic agent to interact more specifically and/or effectively with the wildtype (i.e., “wt”) protein target, thus increasing the therapeutic efficacy of the parental drug and/or decreasing non-specific, potentially deleterious interactions. See, e.g., Hicks, Curr. Med. Chem. 8: 627-50 (2001); Gane and Dean, Curr. Opin. Struct. Biol. 10: 401-4 (2000).

The cell- and animal-based screening methods of the present invention can also be used to assess compositions for their ability to inhibit expression or function of a target polypeptide, thereby affecting, and preferably ameliorating, an aberrant phenotype displayed by an animal expressing mutated Nessie polypeptides. These compositions can include antisense oligonucleotides, RNAi, ribozymes, decoy oligonucleotides, etc.

Antisense oligonucleotides capable of binding polypeptide message can inhibit polypeptide activity by targeting mRNA. Strategies for designing antisense oligonucleotides are well disclosed in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids can be used as antisense oligonucleotides. The antisense oligonucleotides can be of any length suitable for function; for example, in alternative aspects, the antisense oligonucleotides are between about 15 to 100, about 15 to 80, about 15 to 60, about 18 to 40. The optimal length can be determined by routine screening. The antisense oligonucleotides can be present at any concentration. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as disclosed in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold (1995) J. of Biol. Chem. 270:13581-13584).

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but may also be formed in the motif of a hairpin, hepatitis delta virus, group I intron or RNaseP-like RNA (in association with an RNA guide sequence). Examples of such hammerhead motifs are disclosed by Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

RNAi, or “posttranslational gene silencing” refers to methods by which double-stranded RNA molecules trigger a gene silencing response in various cells. In these methods, soluble-stranded RNA molecules are reduced to small interfering RNAs (“siRNAs”), preferably about 21-23 nucleotides in length, by endogenous nucleases. Methods have been disclosed for the design of RNAi oligonucleotides to provide sequence-specific gene silencing. See, e.g., Elbashir et al., Nature 411: 494-8 (2001). The RNAi phenomenon differs from antisense methods, in that it is mediated by double-stranded RNA rather than by single-stranded antisense RNA. Its use has been demonstrated in cells as diverse as those from the nematode C. elegans to numerous mammalian cell types.

RNAi oligonucleotides may be provided to cells either as presynthesized (by either in vitro or in vivo methods) double-stranded RNA molecules, and/or by expressing the RNAi oligonucleotide directly in target cells. For expression of siRNAs within cells, some researchers engineered plasmid vectors that contained either the polymerase III H1-RNA, or U6 promoter, a cloning site for the stem-looped RNA insert, and a 4-5-thymidine transcription termination signal. The inserts were ˜50 nt, with ˜20 nt inverted repeats (coding for the dsRNA stem complementary to a target gene) and ˜10 nt spacers (coding for the loop). Polymerase III promoters were chosen because these promoters generally have well-defined initiation and stop sites and their transcripts lack poly(A) tails. The termination signal for these promoters is defined by 5 thymidines, and the transcript is typically cleaved after the second uridine. Cleavage at this position generates a 3′ UU overhang in the expressed siRNA, which is similar to the 3′ overhangs of synthetic siRNAs. In another approach, U6 promoter-driven expression vectors were made that expressed the sense and antisense strands of siRNAs. Upon expression, these strands presumably anneal in vivo to produce the functional siRNAs. See, e.g., Brummelkamp, et al., Science 296: 550-3 (2002); Paddison et al., Genes and Dev. 16: 948-58 (2002); Paul, et al., Nature Biotechnol. 20: 505-8 (2002); Sui, et al., Proc. Natl. Acad. Sci. USA 99: 5515-20 (2002). Yu, et al., Proc. Natl. Acad. Sci. USA 99: 6047-52 (2002); Miyagishi and Taira, Nature Biotechnol. 20: 497-500 (2002); and Lee, et al., Nature Biotechnol. 20: 500-5 (2002).

“Decoy oligonucleotides” refer to double stranded nucleic acids that bind to a DNA binding protein, thereby preventing binding of the DNA binding protein to its natural target in the cell. Transfection of cis-element double stranded (ds) decoy oligonucleotides has been reported as a powerful tool for gene therapy. See, e.g., Tomita, et al., Exp. Nephrol., 5429-434 (1997). The decoy approach may also enable us to treat diseases by modulation of endogenous transcriptional regulation as a “loss of function” approach at the pre-transcriptional and transcriptional levels in a similar fashion to employing antisense technology as a “loss of function” approach at the transcriptional and translational levels.

Antibodies

The present invention also provides isolated or recombinant antibodies that specifically bind to a polypeptide of the invention. Such antibodies can be used to isolate, identify or quantify a polypeptide of the invention or related polypeptides; and/or as all or part of a therapeutic compositions.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and disclosed in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Polypeptides or peptides can be used to generate antibodies which bind specifically to the polypeptides of the invention. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques disclosed for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

Antibodies generated against the polypeptides of the invention may be used in screening for similar polypeptides from other organisms and samples. In such techniques, polypeptides from the organism are contacted with the antibody and those polypeptides which specifically bind the antibody are detected. Any of the procedures described above may be used to detect antibody binding.

Formulation and Administration of Pharmaceutical Compositions

The various compositions identified or provided according to the foregoing methods may be formulated as pharmaceutical compositions comprising small molecules, nucleic acids, vectors, antibodies and/or polypeptides of the invention. These may preferably be provided to a subject in need thereof, e.g., for treatment or prophylaxis of a disease caused by or related to mutated Nessie polypeptides. As noted herein, animals comprising the mutated nucleic acids and polypeptides of the present invention exhibit altered immune system-related phenotypes. Subjects in need of the pharmaceutical compositions of the present invention include those exhibiting altered levels (e.g., decreases or increases in proliferation) or characteristics (e.g., losses in morphology, function or normal cellular content) in immune system cells as compared to normal “control” animals. Diseases related to such cells include disorders such as autoimmunity, cancers, immunosuppression, etc.

The function of Nessie polypeptides in a subject may be subject to manipulation in a variety of ways to produce a therapeutic effect. For example, people exhibiting impaired immune system function may be appropriate candidates for therapies that target an increase in the amount or function of Nessie polypeptides, thereby altering the phenotype of the subject.

Molecules for therapeutic and/or prophylactic uses can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are disclosed in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions of the invention, when administered orally, can be protected from digestion. This can be accomplished either by complexing, e.g., a nucleic acid, peptide or polypeptide with additional components in a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix (1996) Pharm Res. 13:1760-1764; Samanen (1996) J. Pharm. Pharmacol. 48:119-135; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani (1996) “Systemic delivery of peptides and proteins across absorptive mucosae” Crit. Rev. Ther. Drug Carrier Syst. 13:85-184. For topical, transdermal administration, the agents are formulated into ointments, creams, salves, powders and gels. Transdermal delivery systems can also include, e.g., patches.

Compositions of the invention can also be administered in sustained delivery or sustained release mechanisms, which can deliver the formulation internally. For example, biodegradable microspheres or capsules or other biodegradable polymer configurations capable of sustained delivery of a peptide can be included in the formulations of the invention (see, e.g., Putney (1998) Nat. Biotechnol. 16:153-157).

For inhalation, the nucleic acids, peptides or polypeptides of the invention can be delivered using any system known in the art, including dry powder aerosols, liquids delivery systems, air jet nebulizers, propellant systems, and the like. See, e.g., Patton (1998) Biotechniques 16:141-143; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

Compositions of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are disclosed in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun (1997) Anesth Analg. 85:317-323). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are disclosed in detail, in e.g., Remington's, See also, Bai (1997) J. Neuroimmunol. 80:65-75; Warren (1997) J. Neurol. Sci. 152:31-38; Tonegawa (1997) J. Exp. Med. 186:507-515.

In one aspect, the pharmaceutical formulations comprising nucleic acids, peptides or polypeptides of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble nucleic acids, peptides or polypeptides of the invention have been attached to the surface of the monolayer or bilayer. For example, peptides can be attached to hydrazide-PEG-(distearoylphosphatidyl) ethanolamine-containing liposomes (see, e.g., Zalipsky (1995) Bioconjug. Chem. 6:705-708). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla (1996) J. Pharm. Sci. 85:5-8), transmucosally, or orally. The invention also provides pharmaceutical preparations in which the nucleic acid, peptides and/or polypeptides of the invention are incorporated within micelles and/or liposomes (see, e.g., Suntres (1994) J. Pharm. Pharmacol. 46:23-28; Woodle (1992) Pharm. Res. 9:260-265). Liposomes and liposomal formulations can be prepared according to standard methods and are also well known in the art, see, e.g., Remington's; Akimaru (1995) Cytokines Mol. Ther. 1:197-210; Alving (1995) Immunol. Rev. 145:5-31; Szoka (1980) Ann. Rev. Biophys. Bioeng. 9:467, U.S. Pat. Nos. 4,235,871, 4,501,728 and 4,837,028.

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, etc. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton (1997) “Bioavailability and transport of peptides and peptide drugs into the brain” Peptides 18:1431-1439; Langer (1990) Science 249:1527-1533.

Methods for Detecting Novel Nessie Sequences in Samples from Subjects

Subjects, e.g., humans or animals considered at risk for the presence of a mutated Nessie sequence, can be screened for the occurrence of such mutations using many of the compositions described herein. The presence of mutated Nessie nucleic acid and/or protein sequences may be identified by numerous methods known to those of skill in the art, such as NMR, spectrophotometry, radiography, electrophoresis, capillary electrophoresis, high performance liquid chromatography (HPLC), thin layer chromatography (TLC), various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Examples of suitable samples include those obtained from cells, a biological fluid (such as blood, plasma, serum, urine, bile, saliva, tears, cerebrospinal fluid, aqueous or vitreous humor, or any bodily secretion), a transudate or exudate (e.g., fluid obtained from an abscess or other site of infection or inflammation), a fluid obtained from a joint (e.g., a normal joint or a joint affected by disease such as rheumatoid arthritis, osteoarthritis, gout or septic arthritis), or the like. Preferred biological samples of the present invention are blood, plasma, or serum.

Samples may be obtained from any organ or tissue (including a biopsy or autopsy specimen) or may comprise cells (including primary cells, passaged or cultured primary cells, cell lines, cells conditioned by a specific medium) or medium conditioned by cells. In preferred embodiments, a biological sample is free of intact cells. If desired, the biological sample may be subjected to prior processing, such as lysis, extraction, subcellular fractionation, and the like. See, Deutscher (ed.), Meth. Enzymol. 182:147-238 (1990).

Such methods can be used not only for diagnosis and for prognosis of existing disease, but may also be used to predict the likelihood of the future occurrence of disease, for selecting subjects for inclusion in a clinical trial, or for assessing the effectiveness of a particular treatment regimen.

Animals Comprising Novel Nessie Sequences

The present invention also relates to animals, one or more cells of which comprise a Nessie nucleic acid not ordinarily present in an animal of that species. Such animals might comprise, for example, a Nessie nucleic acid, or a mutated form thereof, obtained from one species and introduced into an animal of a different species, such as a nucleic acid encoding a human Nessie protein inserted into a mouse or vice versa. Alternatively, a nucleic acid encoding a human Nessie protein, or a mutated form thereof, may be inserted into a human, to alter the gene dosage of that particular protein.

In another alternative, endogenous Nessie nucleic acid sequences in an animal may be mutated, e.g., using mutagens such as: radiation (gamma, beta, alpha, UV, etc.); base analogues such as bromouracil and aminopurine; chemicals such as nitrous acid, nitrosoguanidine, ethylnitrosourea, and ethylmethanesulfonate; intercalating agents such as acridine orange and ethidium bromide, as described above to provide animals, one or more cells of which comprise mutated Nessie nucleic acid sequences.

Preferred animal species include primate, caprine, bovine, ovine, porcine, and murine species. Most preferred are mice, due to their small size and (relatively) short gestational period and lifespan, which provide the ability to perform multiple rounds of breeding, while maintaining a relatively small animal facility.

The sequences used to provide transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,258,998; 6,211,428; 6,187,992; 6,156,952; 6,118,044; 6,111,166; 6,107,543; 6,107,541; 6,011,197; 5,959,171; 5,945,577; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., U.S. Pat. No. 6,395,958, and Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, discloses making and using transgenic non-human mammals which express in their brains a nucleic acid construct comprising a DNA sequence. Rideout et al., Cell 109: 17-27 (2002); and Rideout et al., Nat. Genet. 24: 109-10 (2000) disclose nuclear transfer cloning methods may be used to provide animals comprising a mutated sequence of interest, e.g., by transferring the genetic material from a cell harboring the mutated sequence, preferably an embryonic stem cell in mice, into an enucleated oocyte. See, e.g., U.S. Pat. No. 5,387,742, discloses injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, discloses making and using a transgenic mouse whose genome comprises a disruption of the gene encoding amyloid precursor protein (APP).

The animals of the present invention may be used for a variety of purposes, including the production of proteins encoded by expression constructs as described herein, screening methods to identify modulators of Nessie polypeptides, testing the effects of such modulators on altered immune system-related phenotypes, identifying genetic modulators of altered immune system-related phenotypes, etc.

In certain embodiments, such animals are used in sensitized genetic screens. Sensitized screens allow the identification of additional sequences in an animal that relate in some fashion to a gene of interest. In particular, genes and their expressed products that ameliorate or worsen one or more phenotypic characteristics of the mutated Nessie sequences of the present invention can be identified. Such methods can comprise screening for additional mutations on a sensitized genetic background, where the sensitized background exhibits an altered immune system-related phenotype. Genes that are related to such phenotypes may be readily identified using such screens.

The methods described herein for generating and identifying novel Nessie sequences, such as ENU mutagenesis methods, knockout generation, knockin generation, etc., can be applied to such sensitized animals to identify mutations upstream from a mutated Nessie polypeptide in a pathway, downstream in a pathway, or additional mutations in Nessie proteins, that affect a phenotype of interest. As discussed above, both dominant and recessive breeding screens can be used to characterize and identify such mutations.

### EXAMPLES

### Example 1

### ENU Mutagenesis

Male B6 were mutagenized with 3 doses of 85 mg ENU (ethylnitrosourea)/kg body weight by intra-peritoneal injection. After regaining fertility (approximately 3 months) the mutagenized mice (termed G0) were bred with wildtype B6 female mice to produce G1 founder male offspring. The G1 males were bred with wildtype B6 female mice, the offspring referred to as G2 mice. G2 female mice were bred with the G1 male parent to produce approximately 20 G3 offspring. The G3 offspring were phenotyped for outlier mutants. A mutant pedigree was identified when one or more of the G3 offspring exhibit a phenotype not seen in wildtype mice, where a pedigree is a series of G3 mice derived from the same G1 male parent. On identification of a mutant pedigree, the mutation were maintained by breeding to B6 wildtype mice, and were mapped by outcrossing to another mouse strain, where the offspring from this cross are intercrossed and the offspring are phenotyped and genotyped.

### Example 2

### Phenotypic Screening of Mutant Mice

In a library bred from ENU-treated C57BL/6J (B6) mice, a third generation male offspring was identified with low T cells in the peripheral blood. A mutant breeding line, Nessie, had been established by backcrossing this founder to normal B6 mice. Diminished peripheral T cells was inherited as a single recessive Mendelian trait.

### Example 3

### Chromosomal Mapping of Mutations

To map the chromosomal location of the Nessie mutation, B6 Nessie animals were outcrossed to NOD.H2k congenic mice. Progeny from this cross (N1) were intercrossed. Simple sequence length polymorphism (SSLP) typing of tail DNA from NIF1 animals that exhibited low T cells revealed linkage to chromosome 15. The recombinational breakpoints in these animals was further delimited using markers between D15Mit159 and D15Mit172, positioning the Nessie mutation within a 46 megabase (Mb) region. Novel polymorphic markers were identified to further narrow the region containing the Nessie mutation to a 3.6 Mb region that was predicted to contain exons from 39 genes.

### Example 4

### Identification of the Nessie Mutation

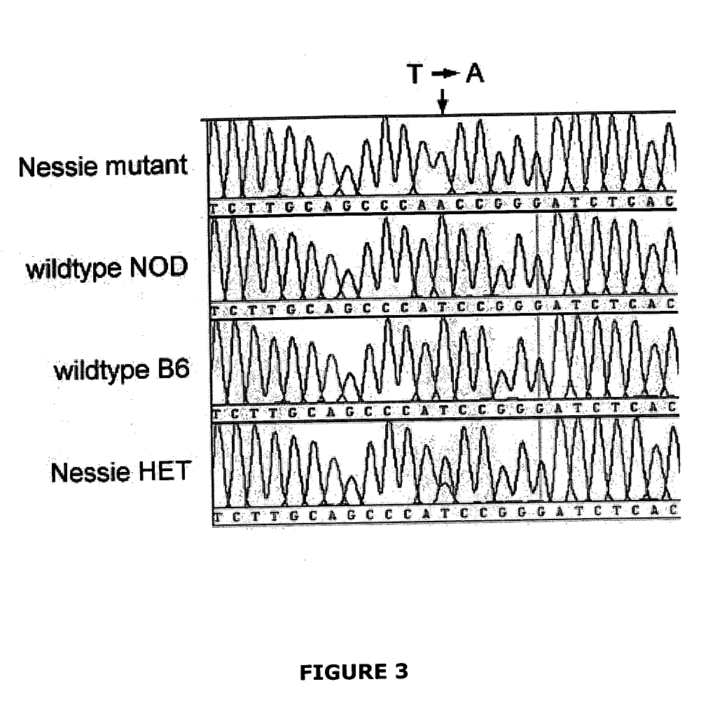

Primers were designed to PCR amplify and sequence genomic DNA covering splice sites and exons of many genes within the critical interval. A single base change was identified in the sequence in exon 1 of the Nessie gene, where the nucleotide ‘T’ had been mutated to ‘A’ (see FIG. 3).

### Example 5

### Cellular Characterization of Nessie Immune System

Various assays have been performed to investigate the immune system development and function in mutant Nessie mice. One example is a bone-marrow reconstitution of irradiated mice that indicates the Nessie phenotype to be intrinsic to Nessie thymocytes and not the thymic environment (see FIG. 4).

### Example 6

### Nessie T-Cell Differentiation Defect

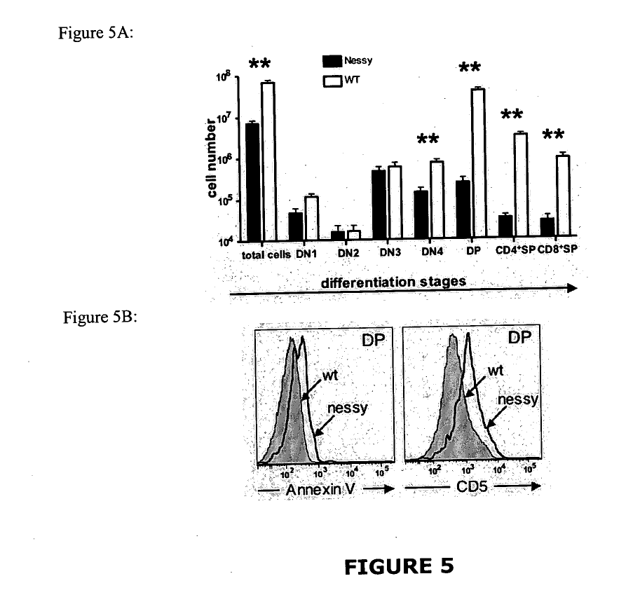

Analysis of variant strain nessie has revealed a defect in T cell differentiation within the thymus, typified by an increased proportion of CD4⁻CD8⁻ double negative (DN) T cell progenitors (FIG. 5A, middle panels), a ten fold reduction in thymocyte numbers, five-fold fewer cells differentiating to the CD4⁻CD8⁻CD44loCD25lo DN4 stage, and only 1% of normal numbers differentiating to the CD4⁺CD8⁺ double positive (DP) stage (FIG. 5B). An increased proportion of DP cells in nes/nes thymi stained with annexin V, consistent with the loss of DP cells through exaggerated apoptosis (FIG. 5C). The nes/nes DP cells displayed increased CD5 (FIG. 5C), an inhibitory receptor whose expression increases in proportion to TCR signal-transduction (Azzam, H. S. et al., 1998, J Exp Med 188:2301-11). The high expression of CD5 and CD44 on nes/nes DP and mature T cells, respectively, resembles mouse mutants with exaggerated TCR signalling due to defects in Cbl (i.e., Casitas B-lineage lymphoma) (Naramura, M., et al., 1998, Proc Natl Acad Sci USA 95:15547-52), slap (i.e., src-like adaptor protein) (Sosinowski, T., et al., 2001, Immunity 15:457-66), and double defects in Cbl and Cblb (Naramura, M. et al., 2002, Nat Immunol 3:1192-9).

In contrast to this profound differentiation defect in the αβ T cell lineage, cells with γδ TCRs were present at twice normal numbers within the thymic DN subset. There were no detected defects in B cell development or peripheral subsets, and nessie homozygous mice were fertile, and appeared healthy in all other respects. The nessie mutation therefore causes a lineage-specific differentiation defect.

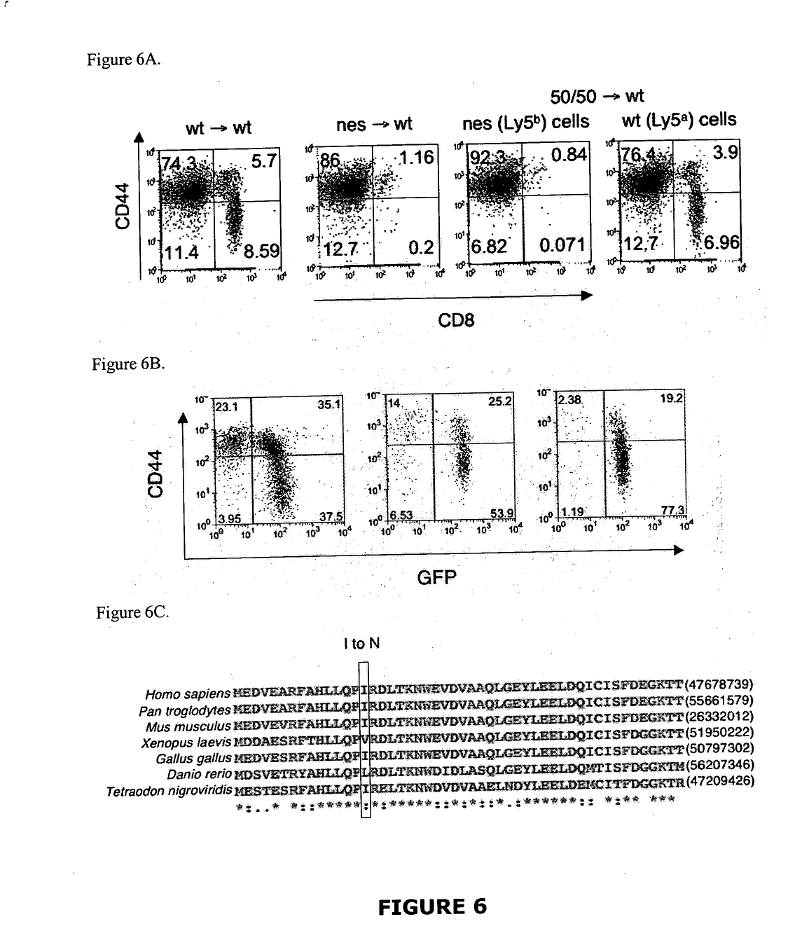

When irradiated mice were reconstituted with a mixture of 50% B6-nes/nes and 50% wild type B6-Ly5^{a} bone marrow, nes/nes-derived T cells were selectively defective in T cell differentiation, accounting for less than 2.5% of thymocytes and 3.3% of splenic T cells, with the latter selectively displaying the characteristic nessie phenotype of high CD44 expression. Normal CD44^{low} T cells were selectively restored, however, in the progeny of nes/nes bone marrow stem cells that had been transduced with a bi-cistronic retroviral vector expressing the wild-type long cDNA isoform of ENSMUSG0000008690 and green fluorescent protein (GFP+T cells in FIG. 6A). The single Ile15Asn substitution, affecting a conserved residue throughout vertebrate species (FIG. 6B), thus causes the T-cell specific differentiation defect in nessie mice.

ENSMUSG0000008690 was annotated as encoding an unknown hypothetical protein in all databases searched, but alignments based on the conserved N and C terminal regions led to its identification as encoding kleisin β (Schleiffer, A. et al., 2003, Mol Cell 11, 571-5), a subunit of the condensin II complex (Ono, T. et al., 2003, Cell 115, 109-21; Yeong, F. M. et al., 2003, Curr Biol 13, 2058-64). Condensins and the closely related cohesins are ubiquitously expressed multiprotein complexes which package chromosomes and hold replicated sister chromatids together between S and M phase of cell cycle in eukaryotes and prokaryotes (Jessberger, R. 2002, Nat Rev Mol Cell Biol 3:767-78). They comprise a V-shaped heterodimer of ATP-cassette SMC proteins closed into a ring by a kleisin subunit, probably encircling individual or sister chromatids. Condensins comprise SMC2 and SMC4 closed by kleisin γ (CAP-H) in condensin I and by kleisin β in condensin II. The other subunits of these complexes are CAP-D2 and CAP-G in condensin I or CAP-D3 (HCP-6) and CAP-G2 (also called FLJ20311 or More Than Blood, MTB) in condensin II (Ono, T. et al., 2003, Cell 115:109-21; Yeong, F. M. et al., 2003, Curr Biol 13:2058-64; Smith, E. D. et al., 2004, Mol Cell Biol 24:1168-73). Condensin II is required for chromosome condensation in early prophase, whereas condensin I affects the timing of progression from prometaphase to anaphase (Ono, T., et al., 2004, Mol Biol Cell 15:3296-308; Hirota, T., et al., 2004, J Cell Sci 117:6435-45). Down-regulation of condensin subunits by siRNA in HeLa cells slowed, but did not prevent chromosome segregation (Ono, T. et al. 2003, Cell 115:109-21; Ono, T., et al., 2004, Mol Biol Cell 15:3296-308; Hirota, T. et al., 2004, J Cell Sci 117:6435-45; Watrin, E. & Legagneux, V., 2005, Mol Cell Biol 25:740-50). Knockouts of SMC2, SMC4, CAP-D2, CAP-G and kleisin γ have been shown to be lethal in several organisms (Jessberger, R., 2002, Nat Rev Mol Cell Biol 3:767-78) as has deletion of the condensin II subunit MTB in mice (Smith, E. D. et al., 2004, Mol Cell Biol 24:1168-73). The nessie T cell differentiation defect does not appear to stem from a mitotic defect, however, since the animals are viable and normal sized, nessie thymocytes proliferate efficiently in stromal cell cultures (data not shown), and CD5 and CD44 over-expression is a well established indicator of chronically elevated TCR signaling.

In invertebrates, additional roles in regulating gene transcription appear to have been added to the condensins. In C. elegans, a condensin homolog mediates X chromosome dosage compensation (reviewed in Jessberger, R., 2002, Nat Rev Mol Cell Biol 3:767-78), and in yeast and Drosophila condensin I subunits have been implicated in silencing of chromosomal regions (Ono, T. et al., 2003, Cell 115:109-21; Bhalla, N., et al., 2003, Mol Biol Cell 13:632-45; Machin, F. et al., 2004, Curr Biol 14:125-30; Lupo, R., et al., 2001, Mol Cell 7:127-36; Dej, K., et al., 2004, Genetics 168:895-906) mediated either through a histone deacetylase (Machin, F. et al., 2004, Curr Biol 14, 125-30) or through a polycomb group protein (Lupo, R., et al., 2001, Mol Cell 7:127-36). In the human Hela cell line, SMC2, SMC4 and CAP-G co-immunoprecipitate with the DNA methylase DNMT3B which also immunoprecipitates SNF2H, a chromatin remodeling enzyme (Geiman, T. M. et al., 2004, Nucleic Acids Res 32:2716-29). Since TCR signalling and thymocyte differentiation depends upon chromatin remodelling (Williams, C. J. et al., 2004, Immunity 20:719-33; Raaphorst, F. M., et al., 2001, Trends Immunol 22:682-90; Gebuhr, T. C. et al., 2003, J Exp Med 198:1937-49; Chi, T. H. et al., 2003, Immunity 19:169-82), the kleisin β point mutation is likely to disrupt a T cell specific chromatin remodeling step while preserving the ancient mitotic functions of condensin II.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

That which is claimed is:

1. An isolated or recombinant nucleic acid comprising a nucleic acid sequence having at least about 70% sequence identity to SEQ ID NO: 1 or SEQ ID NO: 2, wherein said nucleic acid encodes a Nessie polypeptide.

2. An expression cassette comprising a nucleic acid according to claim 1.

3. A vector comprising a nucleic acid according to claim 1.

4. A vector according to claim 3, wherein said vector is a cloning vehicle selected from the group consisting of a recombinant virus, a plasmid, a phage, a phagemid, a cosmid, a fosmid, a bacteriophage, and an artificial chromosome.

5. A transformed cell comprising a vector according to claim 3.

6. A transformed cell according to claim 5, wherein said transformed cell is a bacterial cell, a mammalian cell, a fungal cell, a yeast cell, an insect cell or a plant cell.

7. An antisense oligonucleotide comprising a nucleic acid sequence complementary to or capable of hybridizing under stringent conditions to a nucleic acid sequence having at least about 70% sequence identity to SEQ ID NO: 1 or SEQ ID NO: 2.

8. A double stranded RNA oligonucleotide comprising a nucleic acid sequence complementary to or capable of hybridizing under stringent conditions to a nucleic acid sequence having at least about 70% sequence identity to SEQ ID NO: 1 or SEQ ID NO: 2.

9. An isolated or recombinant polypeptide comprising (i) an amino acid sequence having at least about 80% sequence identity to SEQ ID NO: 5 or SEQ ID NO:6; or (ii) an amino acid sequence encoded by a nucleic acid comprising a sequence having at least about 70% sequence identity to SEQ ID NO: 1 or SEQ ID NO: 2.

10. An isolated or recombinant antibody that specifically binds to a polypeptide according to claim 9.

11. A hybridoma comprising an antibody according to claim 10.

12. A transgenic non-human animal comprising a heterologous nucleic acid, wherein said nucleic acid comprises a sequence having at least about 70% sequence identity to SEQ ID NO: 1 or SEQ ID NO: 2, wherein said animal exhibits an altered immune system phenotype, relative to a wildtype phenotype.

13. The transgenic non-human animal of claim 12, wherein the animal is a mouse or a rat.

14. A cell or cell line derived from a transgenic non-human animal according to claim 13.

15. A knockout non-human animal, wherein an endogenous gene sequence comprising a nucleic acid sequence having at least about 70% sequence identity to SEQ ID NO: 1 or SEQ ID NO: 2 is disrupted so as to produce an altered immune system phenotype, relative to a wildtype phenotype.

16. The knockout non-human animal of claim 15, wherein the animal is a mouse or a rat.

17. A cell or cell line derived from a knockout non-human animal according to claim 15.

18. A non-human mammalian animal comprising a non-naturally occurring mutation in a Nessie gene.

19. A non-human mammalian animal according to claim 18, wherein the resulting mutated Nessie gene causes a deficit in an immune system-related phenotype in said mammalian animal.

20. A non-human mammalian animal according to claim 18, wherein said mutated Nessie gene is expressed as a cDNA having substantial sequence homology with SEQ ID NO: 1 or SEQ ID NO: 2.

21. A non-human mammalian animal according to claim 18, wherein said mutated Nessie gene comprises a mutation in a codon.

22. A non-human mammalian animal according to claim 21, wherein said mutation in a codon is a single nucleotide change in a codon.

23. A non-human mammalian animal according to claim 22, wherein said mutation in the nucleotide codon results in an Isoleucine to Asparagine amino acid change.

24. A non-human mammalian animal according to claim 1, wherein said animal is a mouse expressing a cDNA encoding the protein of SEQ ID NO: 3 or SEQ ID NO: 4.


SEQUENCE LISTING (61 sequences)

SEQ ID NO: 1 - DNA, 1993 bp, Mus musculus
gccagaaccagtagcgggttgagccatggttccgggcttctagtcccgttctagacatggaggatgtggaggtgcgctttgctcacctcttgcagcccatccgggatctcactaagaactgggaggtggacgtggcggcacagctgggcgagtatctggaggagctggaccagatctgcatttcttttgatgaaggcaaaaccacaatgaacttcattgaggcagcactgttgatccagggctcagcctgtgtctacagtaagaaggtggagtacctctactcgctggtctaccaggctctcgattttatttctggcaagaggcgggccaaacagctctccttagttcaggaagatgggagcaagaagactgtcaactcagagactccctgtgaaacagagaatgagttcctgtcactggacgacttccctgactcccgggctaatgtggatctgaaaaatgatcaggcatccagtgagctgcttatcatacccctactgcccatggccctggtggcccctgatgaagtggaaaagaacagcagccccttgtatagctgtcagggtgacatcttggccagccggaaggatttcaggatgaacacgtgtatgcctaaccccagaggctgctttatgttagatccagtgggaatgtgtcctgtggagcctgtggtgcccgtggagccataccccatgtcaaggagccagaaagatcctgaggacgctgaggagcagcccatggaagtgtctaggaacgggagtcctgttcctgtacccgacatctcccaagagccagatggtccagcgctcagcggtggagaggaggatgcagaggatggagcagagcccctggaggttgctctagagcctgcagagccaaggacctcacagcagagtgccatcttgccaaggagatacatgctgcgggaacgacaaggggcaccggagcctgcctcccggctacaggagaccccagacccctggcagagcctggacccttttgactccttggaatctaaggtcttccagaaagggaaaccctattctgtgccacccggtgtggaggaggctccaggacagaagcgcaagaggaagggtgccaccaagttgcaggacttccacaagtggtacctggatgcctatgctgaacaccctgacggcaggagggctcggcggaagggcccaacctttgcagacatggaagtcctgtactggaaacatgtgaaagaacagctcgagacccttcagaagctgcggagacgtaagatcaacgagagatggctacctggggccaagcaggatctgtggcctacagaggaggatcgcttggaagagtccctcgaggacctcggggtagcagatgactttctagagcccgaggagtacgtggaggagcctgcgggggtgatgcccgaggaagctgctgacctcgatgcagaggccatgccagagtccctgagatacgaggagctggtccggagaaatgtggaactcttcattgccacctcccagaagtttatccaggagacagagctgagccaacgcatcagggactgggaagataccatccagcccctgctccaggagcaggagcagcatgtgccctttgatatccatatctacggggaccagttggcttcacggttcccccagctcaatgaatggtgtcccttttcagagcttgtagcagggcagcctgcttttgaggtgtgccgctccatgctggcctccttgcaactggctaatgactacacagtggagatcactcagcagccaggactggaggcagctgtggacacaatgtctctgagactgctcacacaccagcgagcccacacccgcttccagacctatgctgcaccatccatggcccagccttgagtggacagcactgaggcaggggtggaaagtagtatatacctggaggtctttgcccctaatgtgctatggggccattcactccagtgctgcctcctggctggcctagcctaata

SEQ ID NO: 2 - DNA, 1993 bp, Homo sapiens
gaactagtggcgggctgaggacgccgtacccctcggaaggcagccctgcggtccctttgccgcccgttccctcccggacatggaggacgtggaggcgcgcttcgcccacctcttgcagcccatccgcgacctcaccaagaactgggaggtggacgtggcggcccagctgggcgagtatctggaggagctggatcagatctgcatttcttttgacgaaggcaagaccacaatgaacttcattgaggcagcgttgttgatccagggctctgcctgcgtctacagtaagaaggtggaatacctctactcactcgtctaccaggcccttgatttcatctctggaaagaggcgggccaagcagctctcttcggtgcaggaggacagggccaatggggttgccagctccggggtcccccaggaggcagagaatgagttcctgtcgctggatgacttccctgactcccggactaacgtggatctcaagaatgatcagacgcccagtgaggtcctcatcatccccctcctgcccatggccctggtggcccctgatgaaatggagaagaacaacaatcccctgtacagccgtcagggtgaggtcctggccagccggaaggatttcaggatgaacacgtgcgttccccaccccagaggggccttcatgttggagccagagggcatgtcccccatggaaccagcgggcgtttcccccatgccagggacccagaaggacaccgggaggactgaggagcagccaatggaagtttccgtgtgcaggagccctgtcccagcactcggcttctcccaggagccaggcccctctccagaaggcccgatgcccctgggtgggggcgaggacgaggatgcagaggaggcagtagagcttcctgaggcctcggcccccaaggccgctctggagcccaaggagtccaggagcccgcagcagagtgctgccctgcccaggaggtacatgctgcgggagcgggagggggccccagagcctgcatcctgcgtgaaggagactccagacccctggcagagcctggacccctttgactccttggagtctaagcccttcaagaaaggtaggccttactctgtgcccccctgtgtggaggaggctctgggacagaagcgcaagaggaagggcgctgccaagctgcaggacttccaccagtggtacctggctgcctatgcagaccatgccgacagcaggcggcttcggcgaaagggtccgtcctttgcagacatggaggtcctgtactggacacacgtgaaggagcagttggaaactctccggaagctgcagaggagggaggtggctgagcagtggctgcggcctgcagaggaggaccacctggaggattccctggaagacctgggggcagcagatgactttctagagcctgaggagtacatggagcccgagggagcagaccccagggaagccgctgaccttgacgcagtgccgatgtccctgagctacgaggagctggttcgaaggaatgtggagctcttcatcgccacctcccagaagtttgtccaggagacagagctgagccagcgcatcagggactgggaggacacagtgcagcctctgctccaggagcaggagcagcatgtgccctttgacatccacacctatggggaccagctggtctcacggttcccccagctcaatgagtggtgtccctttgcggagctggtggctggccagccggccttcgaggtgtgtcgttccatgctggcctccctgcagctggccaatgactacacagtggagataacccagcagcccgggctggagatggccgtggacaccatgtccctgagactgctcacgcaccagcgagcgcacaagcgcttccagacctacgctgccccctccatggcccagccctgagtggggagcaccgaggcaggggtgggggaatgtgtactgaggagccgtgtgtctgctcctggctggcccggcctaataaagcagtgttgccatctc

SEQ ID NO: 3 - protein, 607 aa, Mus musculus
MEDVEVRFAHLLQPNRDLTKNWEVDVAAQLGEYLEELDQICISFDEGKTTMNFIEAALLIQGSACVYSKKVEYLYSLVYQALDFISGKRRAKQLSLVQEDGSKKTVNSETPCETENEFLSLDDFPDSRANVDLKNDQASSELLIIPLLPMALVAPDEVEKNSSPLYSCQGDILASRKDFRMNTCMPNPRGCFMLDPVGMCPVEPVVPVEPYPMSRSQKDPEDAEEQPMEVSRNGSPVPVPDISQEPDGPALSGGEEDAEDGAEPLEVALEPAEPRTSQQSAILPRRYMLRERQGAPEPASRLQETPDPWQSLDPFDSLESKVFQKGKPYSVPPGVEEAPGQKRKRKGATKLQDFHKWYLDAYAEHPDGRRARRKGPTFADMEVLYWKHVKEQLETLQKLRRRKINERWLPGAKQDLWPTEEDRLEESLEDLGVADDFLEPEEYVEEPAGVMPEEAADLDAEAMPESLRYEELVRRNVELFIATSQKFIQETELSQRIRDWEDTIQPLLQEQEQHVPFDIHIYGDQLASRFPQLNEWCPFSELVAGQPAFEVCRSMLASLQLANDYTVEITQQPGLEAAVDTMSLRLLTHQRAHTRFQTYAAPSMAQP

SEQ ID NO: 4 - protein, 605 aa, Homo sapiens
MEDVEARFAHLLQPNRDLTKNWEVDVAAQLGEYLEELDQICISFDEGKTTMNFIEAALLIQGSACVYSKKVEYLYSLVYQALDFISGKRRAKQLSSVQEDRANGVASSGVPQEAENEFLSLDDFPDSRTNVDLKNDQTPSEVLIIPLLPMALVAPDEMEKNNNPLYSRQGEVLASRKDFRMNTCVPHPRGAFMLEPEGMSPMEPAGVSPMPGTQKDTGRTEEQPMEVSVCRSPVPALGFSQEPGPSPEGPMPLGGGEDEDAEEAVELPEASAPKAALEPKESRSPQQSAALPRRYMLREREGAPEPASCVKETPDPWQSLDPFDSLESKPFKKGRPYSVPPCVEEALGQKRKRKGAAKLQDFHQWYLAAYADHADSRRLRRKGPSFADMEVLYWTHVKEQLETLRKLQRREVAEQWLRPAEEDHLEDSLEDLGAADDFLEPEEYMEPEGADPREAADLDAVPMSLSYEELVRRNVELFIATSQKFVQETELSQRIRDWEDTVQPLLQEQEQHVPFDIHTYGDQLVSRFPQLNEWCPFAELVAGQPAFEVCRSMLASLQLANDYTVEITQQPGLEMAVDTMSLRLLTHQRAHKRFQTYAAPSMAQP

SEQ ID NO: 5 - protein, 607 aa, Mus musculus
MEDVEVRFAHLLQPIRDLTKNWEVDVAAQLGEYLEELDQICISFDEGKTTMNFIEAALLIQGSACVYSKKVEYLYSLVYQALDFISGKRRAKQLSLVQEDGSKKTVNSETPCETENEFLSLDDFPDSRANVDLKNDQASSELLIIPLLPMALVAPDEVEKNSSPLYSCQGDILASRKDFRMNTCMPNPRGCFMLDPVGMCPVEPVVPVEPYPMSRSQKDPEDAEEQPMEVSRNGSPVPVPDISQEPDGPALSGGEEDAEDGAEPLEVALEPAEPRTSQQSAILPRRYMLRERQGAPEPASRLQETPDPWQSLDPFDSLESKVFQKGKPYSVPPGVEEAPGQKRKRKGATKLQDFHKWYLDAYAEHPDGRRARRKGPTFADMEVLYWKHVKEQLETLQKLRRRKINERWLPGAKQDLWPTEEDRLEESLEDLGVADDFLEPEEYVEEPAGVMPEEAADLDAEAMPESLRYEELVRRNVELFIATSQKFIQETELSQRIRDWEDTIQPLLQEQEQHVPFDIHIYGDQLASRFPQLNEWCPFSELVAGQPAFEVCRSMLASLQLANDYTVEITQQPGLEAAVDTMSLRLLTHQRAHTRFQTYAAPSMAQP

SEQ ID NO: 6 - protein, 605 aa, Homo sapiens
MEDVEARFAHLLQPIRDLTKNWEVDVAAQLGEYLEELDQICISFDEGKTTMNFIEAALLIQGSACVYSKKVEYLYSLVYQALDFISGKRRAKQLSSVQEDRANGVASSGVPQEAENEFLSLDDFPDSRTNVDLKNDQTPSEVLIIPLLPMALVAPDEMEKNNNPLYSRQGEVLASRKDFRMNTCVPHPRGAFMLEPEGMSPMEPAGVSPMPGTQKDTGRTEEQPMEVSVCRSPVPALGFSQEPGPSPEGPMPLGGGEDEDAEEAVELPEASAPKAALEPKESRSPQQSAALPRRYMLREREGAPEPASCVKETPDPWQSLDPFDSLESKPFKKGRPYSVPPCVEEALGQKRKRKGAAKLQDFHQWYLAAYADHADSRRLRRKGPSFADMEVLYWTHVKEQLETLRKLQRREVAEQWLRPAEEDHLEDSLEDLGAADDFLEPEEYMEPEGADPREAADLDAVPMSLSYEELVRRNVELFIATSQKFVQETELSQRIRDWEDTVQPLLQEQEQHVPFDIHTYGDQLVSRFPQLNEWCPFAELVAGQPAFEVCRSMLASLQLANDYTVEITQQPGLEMAVDTMSLRLLTHQRAHKRFQTYAAPSMAQP

SEQ ID NO: 7 - DNA, 264 bp, Mus musculus
gcgcgcagtgccctggctacgtacttccggggcgggagcaccaaaatggcgccagaaccagtagcgggttgagccatggttccgggcttctagtcccgttctagacatggaggatgtggaggtgcgctttgctcacctcttgcagcccatccgggatctcactaagaactgggaggtggacgtggcggcacagctgggcgagtatctggaggaggtgagggcggcgggggaatggcatcggcctgggcgctcaggctgcattgc

SEQ ID NO: 8 - DNA, 202 bp, Mus musculus
aggcatgccctcgcttaccgagcccagcttactaatgttctttcttgcagctggaccagatctgcatttcttttgatgaaggcaaaaccacaatgaacttcattgaggcagcactgttgatccagggctcagcctgtgtctacagtaagaaggtgggttccacagaccgccgacagttgtgcttagtgttgagctcctggtg

SEQ ID NO: 9 - DNA, 156 bp, Mus musculus
cccataggtccctcctttccacatccttactgtgtgtttctggttgccaggtggagtacctctactcgctggtctaccaggctctcgattttatttctggcaagaggtgagtattagaagtgagccagaaggaagaggcgtttgttgtcttagctg

SEQ ID NO: 10 - DNA, 185 bp, Mus musculus
gggtctcttgaagcctctcaggatgctcttggtatgtttttctctctcaggcgggccaaacagctctccttagttcaggaagatgggagcaagaagactgtcaactcagagactccctgtgaaacagagaatgaggttagttggggcatgtgaactctgccctgggtgctcgcctggccctgctc

SEQ ID NO: 11 - DNA, 169 bp, Mus musculus
ctgggtgctcgcctggccctgctctcatgagtacttgttgtctcctgcagttcctgtcactggacgacttccctgactcccgggctaatgtggatctgaaaaatgatcaggcatccagtgtgagtgttctggcccttgtccggtggggagagtagtcttagtgctgttc

SEQ ID NO: 12 - DNA, 180 bp, Mus musculus
cttagtgctgttccgtgtagaattcagagctctcccctccctacccacaggagctgcttatcatacccctactgcccatggccctggtggcccctgatgaagtggaaaagaacagcagccccttgtataggtacaacactgagctagcatgggggagaaggggaggacacctgcctggga

SEQ ID NO: 13 - DNA, 255 bp, Mus musculus
acacctgcctgggaagggcgtgcgtagcaagtctgcttttgccaacacagctgtcagggtgacatcttggccagccggaaggatttcaggatgaacacgtgtatgcctaaccccagaggctgctttatgttagatccagtgggaatgtgtcctgtggagcctgtggtgcccgtggagccataccccatgtcaaggagccagaaaggtaagggtttggatgtgggagcttagtgggaaggaagtagtatcagtctc

SEQ ID NO: 14 - DNA, 184 bp, Mus musculus
gaacccttgtaggggcggagctgactctgtctagtgtctgtctctcctagatcctgaggacgctgaggagcagcccatggaagtgtctaggaacgggagtcctgttcctgtacccgacatctcccaagagccaggtgagaagagagtaccagggagactgagctgggtggactccgaccctgga

SEQ ID NO: 15 - DNA, 198 bp, Mus musculus
gcctctttgggctaactggtttcttggttcctgcaccaacatttttctagatggtccagcgctcagcggtggagaggaggatgcagaggatggagcagagcccctggaggttgctctagagcctgcagagccaaggacctcacagcaggtgggacaccaatgaggctgcagagcccaagctgtcaacagagtaagccc

SEQ ID NO: 16 - DNA, 172 bp, Mus musculus
ctgggtggacaggggttggaagaggagaacctgtgggttgtttcttgcagagtgccatcttgccaaggagatacatgctgcgggaacgacaaggggcaccggagcctgcctcccggctacaggtgagaaatgtgaggtccctagacccttcagacccttggcacgtcttcca

SEQ ID NO: 17 - DNA, 167 bp, Mus musculus
gagacctaatgctttccccttctcagcttctacttgtgtttcctgctcaggagaccccagacccctggcagagcctggacccttttgactccttggaatctaaggtcttccagaaaggtaaataggattggtgacaccttgctcgagttgccttgcccagggctcct

SEQ ID NO: 18 - DNA, 208 bp, Mus musculus
tggatcgctttcccttggctactcttgaggcagtccctctctgcctccagggaaaccctattctgtgccacccggtgtggaggaggctccaggacagaagcgcaagaggaagggtgccaccaagttgcaggacttccacaagtggtacctggatgcctgtgagtgagtatatgggctgaggtgacacacccgttttcctgggtgttgg

SEQ ID NO: 19 - DNA, 154 bp, Mus musculus
catcgggcactggggtttcggcctggcgcttgctaactactttctcccagatgctgaacaccctgacggcaggagggctcggcggaagggcccaacctttgcaggtgaggctagggtcctatgtccatgctcagacaacagtctttctcttcct

SEQ ID NO: 20 - DNA, 171 bp, Mus musculus
gtgagcttgggccggatggggaaatccccttataaggtctttgtttgcagacatggaagtcctgtactggaaacatgtgaaagaacagctcgagacccttcagaagctgcggagacgtaaggcaagtacccacaggaccaaggtctgcaaccccacaggtctgaggcatgg

SEQ ID NO: 21 - DNA, 194 bp, Mus musculus
ggtctgaggcatgggatatatgagtttccttacagatgttctctgggcagatcaacgagagatggctacctggggccaagcaggatctgtggcctacagaggaggatcgcttggaagagtccctcgaggacctcggggtagcaggtgggtgcctttagatggaatgtgcgtgtgttgtgtggtcagagtcgtgc

SEQ ID NO: 22 - DNA, 172 bp, Mus musculus
gtactgctggctgagcagcagccttgctctcttctcccatgtctcagcagatgactttctagagcccgaggagtacgtggaggagcctgcgggggtgatgcccgaggaagctgctgacctcggtaggtctgtgatggggagcggcaggaggggagaatgtagccccagagct

SEQ ID NO: 23 - DNA, 156 bp, Mus musculus
gggagaatgtagccccagagctctagcagctcatggttggacctttttagatgcagaggccatgccagagtccctgagatacgaggagctggtccggagaaatgtggtaggcctgggtcaccaaggtgggagcagtggggacatcctgggcccatg

SEQ ID NO: 24 - DNA, 202 bp, Mus musculus
gacatcctgggcccatgtgtgcaagggagaacccactggtccttctgtaggaactcttcattgccacctcccagaagtttatccaggagacagagctgagccaacgcatcagggactgggaagataccatccagcccctgctccaggagcaggtgaggcgtgctattggggactagagcctcctcccaccagcccggggttt

SEQ ID NO: 25 - DNA, 250 bp, Mus musculus
gcccagacctgtctctccacttgaagctaacatgtcctttccctctgcaggagcagcatgtgccctttgatatccatatctacggggaccagttggcttcacggttcccccagctcaatgaatggtgtcccttttcagagcttgtagcagggcagcctgcttttgaggtgtgccgctccatgctggcctccttgcaactggtaagtggcctgggacacaagggatggggcagcggccctggactctactg

SEQ ID NO: 26 - DNA, 355 bp, Mus musculus
caagggatggggcagcggccctggactctactgacaccttgtttccacaggctaatgactacacagtgggagatcactcagcagccaggactggaggcagctgtggacacaatgtctctgagactgctcacacaccagcgagcccacacccgcttccagacctatgctgcaccatccatggcccagccttgagtggacagcactgaggcaggggtggaaagtagtatatacctggaggtctttgcccctaatgtgctatggggccattcactccagtgctgcctcctggctggcctagcctaataaagtgttgctaccccacctgttcaccggacagactatttaaatgagctgc

SEQ ID NO: 27 - DNA, 46 bp, Mus musculus
tttgctcacctcttgcagcccatccgggatctcactaagaactggg

SEQ ID NO: 28 - DNA, 46 bp, Mus musculus
tttgctcacctcttgcagcccaaccgggatctcactaagaactggg

SEQ ID NO: 29 - DNA, 287 bp, Homo sapiens
cgacgccgcgcctacgcattttcctgggcgggaacagcaaaatggcgccagaactagtggcgggctgaggacgccgtacccctcggaaggcagccctgcggtccctttgccgcccgttccctcccggacatggaggacgtggaggcgcgcttcgcccacctcttgcagcccatccgcgacctcaccaagaactgggaggtggacgtggcggcccagctgggcgagtatctggaggaggtaagggcggcgggggagtgacgcggggtgggccggcgggtggggctgcg

SEQ ID NO: 30 - DNA, 202 bp, Homo sapiens
agtgcagactgggccttggattccccctgtctttgtgttgtttcttgcagctggatcagatctgcatttcttttgacgaaggcaagaccacaatgaacttcattgaggcagcgttgttgatccagggctctgcctgcgtctacagtaagaaggtgggccctgcttgacgctggtcttggcattttggtggccagtgggacca

SEQ ID NO: 31 - DNA, 156 bp, Homo sapiens
ggggtgggcccctcctttctgggtcctcactgcctgcatctggtcaccaggtggaatacctctactcactcgtctaccaggcccttgatttcatctctggaaagaggtgagttctgcagccactcactgtgctgccctgcatgtggccagggaggc

SEQ ID NO: 32 - DNA, 185 bp, Homo sapiens
ggaggcccctgcagctcctgggatgcccacgggatgtgcttctctctcaggcgggccaagcagctctcttcggtgcaggaggacagggccaatggggttgccagctccggggtcccccaggaggcagagaatgaggtgagtttctttggcatgtggtccccgcccactgtgtgtttgcctgggct

SEQ ID NO: 33 - DNA, 169 bp, Homo sapiens
cactgtgtgtttgcctgggctccgcccccacgagctctgtctccctccagttcctgtcgctggatgacttccctgactcccggactaacgtggatctcaagaatgatcagacgcccagtgtgagtcctggcctggcccctcttaggctggggtgaggtcagcactttcc

SEQ ID NO: 34 - DNA, 180 bp, Homo sapiens
ccactggagtggaagggtgcctggctcacccacccttggcctccatgcaggaggtcctcatcatccccctcctgcccatggccctggtggcccctgatgaaatggagaagaacaacaatcccctgtacaggtagggatctgagcccagcgacggggagggaggcctgcctgggaagggtc

SEQ ID NO: 35 - DNA, 246 bp, Homo sapiens
aggcctgcctgggaagggtctctacagcaggcgtgtttttgccagcacagccgtcagggtgaggtcctggccagccggaaggatttcaggatgaacacgtgcgttccccaccccagaggggccttcatgttggagccagagggcatgtcccccatggaaccagcgggcgtttcccccatgccagggacccagaagggtgagggcttggatgcggggggcttggtgggaaggaagggagggtcttct

SEQ ID NO: 36 - DNA, 184 bp, Homo sapiens
catcttggagaggggctgggctgaccttgtctgatccctgtctctcccagacaccgggaggactgaggagcagccaatggaagtttccgtgtgcaggagccctgtcccagcactcggcttctcccaggagccaggtgagaagagagctccccggtgggactggcagggcagccaaagaggggac

SEQ ID NO: 37 - DNA, 231 bp, Homo sapiens
tctggtctcggcactccttggagctgatcactctcttgctccctgcctaggcccctctccagaaggcccgatgcccctgggtgggggcgaggacgaggatgcagaggaggcagtagagcttcctgaggcctcggcccccaaggccgctctggagcccaaggagtccaggagcccgcagcaggtgggacccacatggaggcctgcagaacctgagctgtgaactggcaaccc

SEQ ID NO: 38 - DNA, 172 bp, Homo sapiens
ccttgaggggagaaagaggagaagtggggaccctgtgactgtctttgcagagtgctgccctgcccaggaggtacatgctgcgggagcgggagggggccccagagcctgcatcctgcgtgaaggtaggagtgttggggccctgacccccggcagggagggatgggcggattcg

SEQ ID NO: 39 - DNA, 167 bp, Homo sapiens
cggctgtgcaccccctacttctccagcgccttcttgtgctctgtgcccaggagactccagacccctggcagagcctggacccctttgactccttggagtctaagcccttcaagaaaggtaattgggtggaaggtacctccactcaggtacccctggctgggtttcct

SEQ ID NO: 40 - DNA, 208 bp, Homo sapiens
ttgcaccctgatcccccagcggctctaagacagtccctgtttgcccccaggtaggccttactctgtgcccccctgtgtggaggaggctctgggacagaagcgcaagaggaagggcgctgccaagctgcaggacttccaccagtggtacctggctgcctgtgagtgggtgtggtgtgcactccggaccactgggagctgggggctgggc

SEQ ID NO: 41 - DNA, 154 bp, Homo sapiens
tgctgtgggcagctcttggcctggctggtgctgagcatgttctttcacagatgcagaccatgccgacagcaggcggcttcggcgaaagggtccgtcctttgcaggtgaggctgaagtcctcggggaagacagttttactctccttcccctacct

SEQ ID NO: 42 - DNA, 171 bp, Homo sapiens
cacggggcctgggaaggacgagggagccctcacaaggcctttgtctgcagacatggaggtcctgtactggacacacgtgaaggagcagttggaaactctccggaagctgcagaggagggaggcaagtcccagctggtcagctgtgatctaggaccccgtggtggccctgat

SEQ ID NO: 43 - DNA, 173 bp, Homo sapiens
ggccctgatgggaggtgacagtgcccctcacagatgctttctctggacaggtggctgagcagtggctgcggcctgcagaggaggaccacctggaggattccctggaagacctgggggcagcaggtgggtgcctgccagggggtggggtggggcttggcacctgccgactagct

SEQ ID NO: 44 - DNA, 169 bp, Homo sapiens
acctgccgactagctgcctgcgtgctgttcactctcccacctcccagcagatgactttctagagcctgaggagtacatggagcccgagggagcagaccccagggaagccgctgaccttggtaggtgggcagcgggctaggagtgctgaggggccactggagctgggggc

SEQ ID NO: 45 - DNA, 150 bp, Homo sapiens
ggggagagcgtggccccttagctgcccagctcacagctaccccttcccagacgcagtgccgatgtccctgagctacgaggagctggttcgaaggaatgtggtaggcctgggttagagggagacggggaggggagggggacaggtgagcgg

SEQ ID NO: 46 - DNA, 202 bp, Homo sapiens
tgcccaggcccctgcttgggaggcagtagctcctgctgatcctcccctaggagctcttcatcgccacctcccagaagtttgtccaggagacagagctgagccagcgcatcagggactgggaggacacagtgcagcctctgctccaggagcaggtgaggcggggccgctgggaaccagagctgtgtgccacgggtctgtccag

SEQ ID NO: 47 - DNA, 250 bp, Homo sapiens
gtccagggccttgcctctctccgcagccaacatgcccctcccctgtgcaggagcagcatgtgccctttgacatccacacctatggggaccagctggtctcacggttcccccagctcaatgagtggtgtccctttgcggagctggtggctggccagccggccttcgaggtgtgtcgttccatgctggcctccctgcagctggtgagtagcctgggatacgtgggagggggagacggtccccagaccctgct

SEQ ID NO: 48 - DNA, 337 bp, Homo sapiens
gtgggagggggagacggtccccagaccctgctgatgtgccacccctgcaggccaatgactacacagtggagataacccagcagcccgggctggagatggccgtggacaccatgtccctgagactgctcacgcaccagcgagcgcacaagcgcttccagacctacgctgccccctccatggcccagccctgagtggggagcaccgaggcaggggtgggggaatgtgtactgaggagccgtgtgtctgctcctggctggcccggcctaataaagcagtgttgccatctcatcttccccctaaaaacccttttatgtacacctgcgcagagaagagggct

SEQ ID NO: 49 - DNA, 46 bp, Homo sapiens
ttcgcccacctcttgcagcccatccgcgacctcaccaagaactggg

SEQ ID NO: 50 - DNA, 46 bp, Homo sapiens
ttcgcccacctcttgcagcccaaccgcgacctcaccaagaactggg

SEQ ID NO: 51 - DNA, 25 bp, Mus musculus
tcttgcagcccaaccgggatctcac

SEQ ID NO: 52 - DNA, 25 bp, Mus musculus
tcttgcagcccatccgggatctcac

SEQ ID NO: 53 - DNA, 25 bp, Mus musculus
tcttgcagcccatccgggatctcac

SEQ ID NO: 54 - DNA, 25 bp, Mus musculus
tcttgcagcccatccgggatctcac

SEQ ID NO: 55 - protein, 50 aa, Homo sapiens
MEDVEARFAHLLQPIRDLTKNWEVDVAAQLGEYLEELDQICISFDEGKTT

SEQ ID NO: 56 - protein, 50 aa, Pan troglodytes; identical to SEQ ID NO: 55

SEQ ID NO: 57 - protein, 50 aa, Mus musculus
MEDVEVRFAHLLQPIRDLTKNWEVDVAAQLGEYLEELDQICISFDEGKTT

SEQ ID NO: 58 - protein, 50 aa, Xenopus laevis
MDDAESRFTHLLQPVRDLTKNWEVDVAAQLGEYLEELDQICISFDGGKTT

SEQ ID NO: 59 - protein, 50 aa, Gallus gallus
MEDVESRFAHLLQPIRDLTKNWEVDVAAQLGEYLEELDQICISFDGGKTT

SEQ ID NO: 60 - protein, 50 aa, Danio rerio
MDSVETRYAHLLQPLRDLTKNWDIDLASQLGEYLEELDQMTISFDGGKTM

SEQ ID NO: 61 - protein, 50 aa, Tetraodon nigroviridis
MESTESRFAHLLQPIRELTKNWDVDVAAELNDYLEELDEMCITFDGGKTR